SEQUENCE LISTING (39 sequences)

SEQ ID NO: 1 - DNA, 17 bp, Artificial sequence
  Synthetic primer
caatgccacgcttctgc

SEQ ID NO: 2 - DNA, 20 bp, Artificial sequence
  Synthetic primer
ttctacacacccaagacccg

SEQ ID NO: 3 - DNA, 20 bp, Artificial sequence
  synthetic primer
gccataaggcatcattggac

SEQ ID NO: 4 - DNA, 20 bp, Artificial sequence
  Synthetic primer
aacaacagcctgccacctta

SEQ ID NO: 5 - DNA, 20 bp, Artificial sequence
  synthetic primer
aaggcacactacatcgaggc

SEQ ID NO: 6 - DNA, 19 bp, Artificial sequence
  sythetic primer
cacgctgcgactcttttcc

SEQ ID NO: 7 - DNA, 20 bp, Artificial Sequence
  Synthetic primer
ctgcccaccctcaacttctc

SEQ ID NO: 8 - DNA, 21 bp, Artificial Sequence
  Synthetic primer
gcaggatcgactcgtgtttgt

SEQ ID NO: 9 - DNA, 20 bp, Artificial Sequence
  Synthetic primer
acatgtggcagtgttgcatt

SEQ ID NO: 10 - DNA, 21 bp, Artificial Sequence
  Synthetic primer
tcattgtgctctctgttgcat

SEQ ID NO: 11 - DNA, 20 bp, Artificial Sequence
  Synthetic primer
tgtagaacttgtgggggagg

SEQ ID NO: 12 - DNA, 19 bp, Artificial Sequence
  synthetic primer
ggagggaagtccactctcg

SEQ ID NO: 13 - DNA, 20 bp, Artificial Sequence
  synthetic primer
gcccacgaaagttatgagga

SEQ ID NO: 14 - DNA, 20 bp, Artificial Sequence
  synthetic primer
aaggagatgaccagcctcag

SEQ ID NO: 15 - DNA, 18 bp, Artificial Sequence
  synthetic primer
atgctggacgacagagcc

SEQ ID NO: 16 - DNA, 20 bp, Artificial Sequence
  synthetic primer
ccttcttcaggtcctcctcc

SEQ ID NO: 17 - DNA, 21 bp, Artificial Sequence
  synthetic primer
ggtgcagaaatggcgagaata

SEQ ID NO: 18 - DNA, 21 bp, Artificial Sequence
  synthetic primer
ccggttgcagaacaagtctgt

SEQ ID NO: 19 - DNA, 22 bp, Artificial Sequence
  synthetic primer
gagacccagggtagtgatttga

SEQ ID NO: 20 - DNA, 23 bp, Artificial Sequence
  synthetic primer
aaaagtaatcgaaagccaagcac

SEQ ID NO: 21 - DNA, 22 bp, Artificial Sequence
  synthetic primer
atgagttccttcagctacgagc

SEQ ID NO: 22 - DNA, 20 bp, Artificial Sequence
  synthetic primer
ctgggcatcaacgatccaga

SEQ ID NO: 23 - DNA, 21 bp, Artificial Sequence
  synthetic primer
gctcttcgaggaattgggcaa

SEQ ID NO: 24 - DNA, 23 bp, Artificial Sequence
  synthetic primer
cctctgagatgctgtcatgtagt

SEQ ID NO: 25 - DNA, 26 bp, Artificial Sequence
  synthetic primer
cctttggcacaatgaagtgggtaacc

SEQ ID NO: 26 - DNA, 25 bp, Artificial Sequence
  synthetic primer
cagcagtcagccatttcaccatagg

SEQ ID NO: 27 - DNA, 23 bp, Artificial Sequence
  synthetic primer
cctctgagatgctgtcatgtagt

SEQ ID NO: 28 - DNA, 23 bp, Artificial Sequence
  synthetic primer
ggtgatgtagcgactgtagtgaa

SEQ ID NO: 29 - DNA, 27 bp, Artificial Sequence
  synthetic primer
tgtactgcagagataagtttagctgac

SEQ ID NO: 30 - DNA, 23 bp, Artificial Sequence
  synthetic primer
ccttgtaagtggcttcttgaaca

SEQ ID NO: 31 - DNA, 20 bp, Artificial Sequence
  synthetic primer
gggagaacgagtgagaagcg

SEQ ID NO: 32 - DNA, 20 bp, Artificial Sequence
  synthetic primer
tgcgggtctttcagtacctg

SEQ ID NO: 33 - DNA, 21 bp, Artificial Sequence
  Synthetic primer
cagttctgggagagaagagac

SEQ ID NO: 34 - DNA, 21 bp, Artificial sequence
  Synthetic primer
gggctggattctgttcccata

SEQ ID NO: 35 - DNA, 21 bp, Artificial sequence
  Synthetic shRNA
gcgcgatcacatggtcctgct

SEQ ID NO: 36 - DNA, 21 bp, Artificial sequence
  Synthetic shRNA
caacgagaactccaattctaa

SEQ ID NO: 37 - DNA, 21 bp, Artificial sequence
  Synthetic shRNA
gagcaccttcacaagaatgaa

SEQ ID NO: 38 - DNA, 23 bp, Artificial sequence
  Synthetic gRNA
tcggagcttcgtgggacccgcgg

SEQ ID NO: 39 - DNA, 23 bp, Artificial sequence
  Sythetic gRNA
ccacgaggttggctgacatgggg

(19) United States
(12) Patent Application Publication
(10) Pub. No.: US 2023/0014430 A1
(43) Pub. Date: Jan. 19, 2023

(54) Methods and Compositions for Generating Functionally Mature Beta Cells and Uses Thereof

(71) Applicant: Washington University, St. Louis, MO (US)

(72) Inventors: Jeffrey Millman, St. Louis, MO (US); Daniel Veronese Paniagua, St. Louis, MO (US); Kristina Wernes, St. Louis, MO (US); Leonardo Velazco-Cruz, St. Louis, MO (US)

(73) Assignee: Washington University, St. Louis, MO (US)

(21) Appl. No.: 17/778,079

(22) PCT Filed: Nov. 20, 2020
(86) PCT No.: PCT/US2020/061572

§ 371 Date: May 19, 2022

Related U.S. Application Data
(60) Provisional application No. 62/937,825, filed on Nov. 20, 2019.

(51) Int. Cl. C12N 5/071; A61K 35/39

(52) U.S. Cl. CPC C12N 5/0676; A61K 35/39; C12N 2500/90; C12N 2501/385; C12N 2501/727

(57) ABSTRACT

Among the various aspects of the present disclosure is the provision of methods and compositions for the generation of functionally mature beta cells having enhanced SIX2+ activity and therapeutic benefit and uses thereof. An aspect of the present disclosure provides for a method of generating SIX2-enhanced SC-β cells. In some embodiments, the method comprises providing a population of SC-β cells (or EP cells); providing a SIX2 positive regulator; and/or incubating the population of SC-β cells and the SIX2 positive regulator.

### CROSS-REFERENCE TO RELATED APPLICATIONS

This application claims priority from U.S. Provisional Application Ser. No. 62/937,825 filed on 20 Nov. 2019, which is incorporated herein by reference in its entirety.

### STATEMENT REGARDING FEDERALLY SPONSORED RESEARCH OR DEVELOPMENT

This invention was made with government support under DK114233 awarded by the National Institutes of Health. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention. The subject matter of the Sequence Listing is incorporated herein by reference in its entirety.

### FIELD OF THE INVENTION

The present disclosure generally relates to methods and compositions for the generation of functionally mature beta cells.

### SUMMARY OF THE INVENTION

Among the various aspects of the present disclosure is the provision of methods and compositions for the generation of functionally mature beta cells and uses thereof.

An aspect of the present disclosure provides for a method of generating SIX2-enhanced SC-β cells. In some embodiments, the method comprises providing a population of SC-β cells (or EP cells); providing a SIX2 positive regulator; and/or incubating the population of SC-β cells and the SIX2 positive regulator. In some embodiments, the population of SC-β cells or EP cells and the SIX2 positive regulator are incubated in (in fluid contact with) an effective amount of the SIX2 positive regulator and for an amount of time sufficient to form an increased population of SIX2-enhanced SC-β cells or a population of SIX2-enhanced SC-β cells having increased SIX2 expression, function, or activity compared to the population of SC-β cells not in fluid contact with a SIX2 positive regulator.

In some embodiments, the population of SC-β cells are generated comprising the steps of: providing a stem cell; providing serum-free media; contacting the stem cell with a TGFβ/Activin agonist or a glycogen synthase kinase 3 (GSK) inhibitor or WNT agonist for an amount of time sufficient to form a definitive endoderm cell; contacting the definitive endoderm cell with a FGFR2b agonist for an amount of time sufficient to form a primitive gut tube cell; contacting the primitive gut tube cell with an RAR agonist, and optionally a rho kinase inhibitor, a smoothened antagonist, a FGFR2b agonist, a protein kinase C activator, or a BMP type 1 receptor inhibitor for an amount of time sufficient to form an early pancreas progenitor cell; incubating the early pancreas progenitor cell for at least about 3 days and optionally contacting the early pancreas progenitor cell with a rho kinase inhibitor, a TGF-β/Activin agonist, a smoothened antagonist, an FGFR2b agonist, or a RAR agonist for an amount of time sufficient to form a pancreatic progenitor cell; contacting the pancreatic progenitor cell with an Alk5 inhibitor, a gamma secretase inhibitor, SANT1, Erbb1 (EGFR) or Erbb4 agonist, or a RAR agonist for an amount of time sufficient to form an endocrine progenitor cell; and/or allowing the endocrine progenitor (EP) cell to mature for an amount of time sufficient to form an SC-β cell.

In some embodiments, the effective amount of the SIX2 positive regulator increases differentiation efficiency of the population of SC-β cells into mature SIX2-enhanced SC-β cells capable of biphasic insulin secretion in response to glucose.

In some embodiments, the effective amount of the SIX2 positive regulator results in SIX2-enhanced SC-β cell exhibiting an increased fraction of C-peptide+ SC-β cells compared to the fraction of C-peptide+ SC-β cells not incubated with a SIX2 positive regulator.

In some embodiments, the effective amount of the SIX2 positive regulator results in SIX2-enhanced SC-β cell exhibiting an increased fraction of C-peptide+/NKX6-1+ SC-β cells compared to the fraction of C-peptide+/NKX6-1+ SC-β cells not incubated with a SIX2 positive regulator.

In some embodiments, the effective amount of the SIX2 positive regulator results in improved glucose responsiveness compared to a SC-β cell not incubated with a SIX2 positive regulator.

In some embodiments, the effective amount of the SIX2 positive regulator results in improved calcium coupling compared to a SC-β cell not incubated with a SIX2 positive regulator.

In some embodiments, the effective amount of the SIX2 positive regulator results in improved mitochondrial respiration compared to a SC-β cell not incubated with a SIX2 positive regulator.

In some embodiments, the effective amount of the SIX2 positive regulator results in improved insulin gene expression compared to a SC-β cell not incubated with a SIX2 positive regulator.

In some embodiments, the effective amount of the SIX2 positive regulator results in improved insulin content compared to a SC-β cell not incubated with a SIX2 positive regulator.

In some embodiments, the effective amount of the SIX2 positive regulator results in improved glucose-insulin coupling compared to a SC-β cell not incubated with a SIX2 positive regulator.

In some embodiments, the effective amount of the SIX2 positive regulator results in increased biphasic glucose-stimulated insulin secretion compared to a SC-β cell not incubated with a SIX2 positive regulator.

In some embodiments, the effective amount of the SIX2 positive regulator results in increased glucose-stimulated insulin secretion compared to a SC-β cell not incubated with a SIX2 positive regulator.

In some embodiments, the effective amount of the SIX2 positive regulator results in increased first and second phase insulin secretion compared to a SC-β cell not incubated with a SIX2 positive regulator.

In some embodiments, the effective amount of the SIX2 positive regulator results in increased insulin gene expression and insulin content compared to a SC-β cell not incubated with a SIX2 positive regulator.

In some embodiments, the effective amount of the SIX2 positive regulator results in decreased glucose stimulated calcium flux compared to a SC-β cell not incubated with a SIX2 positive regulator.

In some embodiments, the effective amount of the SIX2 positive regulator results in increased mitochondrial or metabolic respiration compared to a SC-β cell not incubated with a SIX2 positive regulator.

In some embodiments, the effective amount of the SIX2 positive regulator results in increased insulin secretion in response to β cell secretagogues compared to a SC-β cell not incubated with a SIX2 positive regulator.

In some embodiments, the effective amount of the SIX2 positive regulator results in improved β cell health compared to a SC-β cell not incubated with a SIX2 positive regulator.

In some embodiments, the effective amount of the SIX2 positive regulator results in decreased oxidative stress compared to a SC-β cell not incubated with a SIX2 positive regulator.

In some embodiments, the effective amount of the SIX2 positive regulator results in increased protection against cellular stress compared to a SC-β cell not incubated with a SIX2 positive regulator.

In some embodiments, the effective amount of the SIX2 positive regulator results in decreased endoplasmic reticulum (ER) stress compared to a SC-β cell not incubated with a SIX2 positive regulator.

In some embodiments, the effective amount of the SIX2 positive regulator results in improved resistance to ER-mediated cell death compared to a SC-β cell not incubated with a SIX2 positive regulator.

In some embodiments, the SIX2 positive regulator promotes maturation of the SC-β cell transcriptome.

In some embodiments, the SIX2 positive regulator is selected from the group consisting of a TGFβ agonist, a glycogen synthase kinase 3 (GSK-3) inhibitor; a cocaine- and amphetamine-regulated transcript (CART) peptide fragment; an FGFR inhibitor; or a p38 MAPK inhibitor; or combinations thereof.

In some embodiments, the TGFβ agonist is TGFβ1 or TGFβ2. In some embodiments, the GSK-3 inhibitor is CHIR99021. In some embodiments, the CART peptide fragment is CART 62-76 or CART 55-102. In some embodiments, the FGFR inhibitor is AZD4547. In some embodiments, the p38 MAPK inhibitor is doramapimod.

In some embodiments, the stem cell is a HUES8 embryonic stem cell.

In some embodiments, the method further comprises plating the SIX2-enhanced SC-β cells, wherein the SIX2-enhanced SC-β cells are passaged by single cell dispersion prior to plating.

In some embodiments, the plated single β cells are capable of glucose-stimulated insulin secretion.

In some embodiments, the method further comprises transplanting the SIX2-enhanced SC-β cells into a subject in need thereof.

Another aspect of the present disclosure provides for a method of treating a subject in need thereof comprising administering or transplanting a therapeutically effective amount of SIX2-enhanced stem cell-derived beta cells (SC-β cells) to the subject.

In some embodiments, the subject has diabetes.

In some embodiments, the subject has type 2 diabetes (T2D).

Yet another aspect of the present disclosure provides for a method of treating a subject in need thereof comprising transplanting SC-β cells to the subject. In some embodiments, SIX2 is activated after transplantation comprising administering a SIX2 positive regulator to the subject after the SC-β cells are transplanted into the subject.

Yet another aspect of the present disclosure provides for a method of treating a subject in need thereof comprising: administering to a subject a therapeutically effective amount of a SIX2 positive regulator.

In some embodiments, the subject has diabetes.

In some embodiments, the subject has type 2 diabetes (T2D).

Yet another aspect of the present disclosure provides for a method of screening therapeutic agents comprising: providing a SIX2-enhanced SC-β cell; and introducing a compound or composition in fluid contact with the SIX2-enhanced SC-β cell, resulting in a treated SIX2-enhanced SC-β cell; and optionally, testing the SIX2-enhanced SC-β cell function or activity or measuring an amount of glucose stimulated insulin production.

In some embodiments, the method comprises plating the SIX2-enhanced SC-β cells prior to introducing the compound or composition, wherein the SIX2-enhanced SC-β cells are passaged by single cell dispersion prior to plating.

Yet another aspect of the present disclosure provides for a SIX2-enhanced SC-β cell having increased SIX2 expression, function, or activity compared to an SC-β cell not treated with a SIX2 positive regulator.

Yet another aspect of the present disclosure provides for a SIX2-enhanced SC-β cell having increased SIX2 expression, function, or activity compared to an SC-β cell not treated with a SIX2 positive regulator produced according to the any one of the preceding aspects or embodiments.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. SIX2 Controls Glucose-Stimulated Insulin Secretion in Human SC-β Cells. (A) Schematic of hESC differentiation process. (B) Real-time PCR measurements of SIX2 in undifferentiated hESCs and at the end of each stage of the differentiation. Data are presented as the fold change relative to stage 6 cells. n=3. (C) Real-time PCR measurements of SIX2 as a function of time in stage 6 plotted against insulin secretion of sampled cells placed in 20 mM glucose for 1 h. n=4. (D) Dynamic glucose-stimulated insulin secretion of stage 6 cells transfected with control shRNA (sh-ctrl; n=3) or shRNA targeting SIX2 (sh-SIX2-1; n=4). Cells are perfused with 2 mM glucose, except when indicated, in a perifusion chamber. (E) Static glucose-stimulated insulin secretion of sh-ctrl or sh-SIX2-1 transduced stage 6 cells. n=4. (F) Dynamic glucose-stimulated insulin secretion of wild-type (WT) (n=4), KO-SIX2-1 (n=3 technical replicates), or KO-SIX2-2 (n=3 technical replicates) stage 6 cells. (G) Static glucose-stimulated insulin secretion of WT, KO-SIX2-1, or KO-SIX2-2 stage 6 cells. n=4. All data in (B)-(E) were generated with cells from protocol 1 and all data in (F) and (G) were generated with cells from protocol 2. *p<0.05, **p<0.01, ****p<0.0001 by 2-way paired (for low-high glucose comparison) or unpaired (for high-high glucose comparison) t test. Error bars represent s.e.m. See also FIG. 5.

FIG. 2. Subtypes of Differentiated Stage 6 Cells Express SIX2. (A) Immunostaining of SIX2 with the β cell markers NKX6-1 and C-peptide at the end of stages 5 (left) and 6 (right). (B) Flow cytometric quantification of co-expression of C-peptide with SIX2. n=4. (C) Immunostaining of SIX2 with a panel of pancreatic markers at the end of stage 6 with the exception of NGN3/SIX2, which was stained 3 days into stage 5. (D) Flow cytometric quantification of stage 6 cells staining for C-peptide, NKX6.1, and chromogranin A using sh-ctrl and sh-SIX2-1 transduced cells. n=6. ***p<0.001 by 2-way unpaired t test. (E) Schematic summary of marker progression in stages 5 and 6. Scale bar, 25 μm. Error bars represent s.e.m. See also FIG. 6.

FIG. 3. SIX2 Regulates Important β Cell Genes and Gene Sets. (A) Heatmap of 1,000 most differentially expressed genes between stage 6 cells transduced with sh-ctrl and sh-SIX2-1 by p value. n=6. (B) Volcano plot showing all differentially expressed genes. Genes with at least a 2-fold change (FC) are in black. Genes of particular interest are highlighted. (C) Selected enriched gene sets for important β cell processes from the Molecular Signatures Database. Also included 2 custom gene sets comprising 76 genes identified in Veres et al. (2019) and the top 424 genes identified in Nair et al. (2019) positively correlating with time and maturation in vitro. NES, normalized enrichment score. (D) Enrichment plots from the shown gene sets. (E) FCs from genes within enriched β cell-related gene sets. See also FIG. 7, Related to FIG. 3.

FIG. 4. SIX2 Affects Insulin Content, Mitochondrial Respiration, Cytoplasmic Calcium Flux, and Response to Secretagogues in SC-β cells. (A) Insulin content for stage 6 cells. n=12. ****p<0.0001 by 2-way unpaired t test. (B) Proinsulin:insulin content ratio for stage 6 cells. n=12. ns (non-significant) by 2-way unpaired t test. (C) Real-time PCR measurements of INS gene expression for stage 6 cells. n=4. *p<0.05 by 2-way unpaired t test. (D) OCR measurements under basal conditions and after sequential injections of oligomycin (OM), carbonyl cyanide-4-(trifluoromethoxy) phenylhydrazone (FCCP), and antimycin A with rotenone (AA/R). n=10 for sh-ctrl and n=9 for sh-SIX2-1. (E) Calculated OCR:ECAR ratio under basal conditions. n=10 for sh-ctrl and n=9 for sh-SIX2-1. ****p<0.0001 by 2-way unpaired t test. (F) Cytosolic calcium signaling in response to high glucose (20 mM) and high KCl (30 mM) treatment relative to low glucose (2 mM, Fo) for Fluo-4 AM. Violin plots show distribution of cellular responses for sh-ctrl (n=232) and sh-SIX2-1 (n=276) transduced cells with median and quartiles marked with dashed lines. ****p<0.0001 by 2-way unpaired t test. (G) Static glucose-stimulated insulin secretion with cells with 2 mM glucose, 20 mM glucose, or 20 mM glucose with the indicated compound. n=3. ns, ** or ^{†\}p<0.01, *** or ^{\\\}p<0.001, **** or ^{\\\\}p<0.0001 by 2-way unpaired t test. * indicates comparison within same compound treatment. t indicates comparison with low glucose with same shRNA treatment. Error bars represent s.e.m. See also FIG. 8.

FIG. 5. Validation of SIX2 KD and KO assessments. Related to FIG. 1. (A) Real-time PCR measurements of SIX2 gene expression for Stage 6 cells transduced with sh-ctrl, sh-SIX2-1, or sh-SIX2-2 for differentiated HUES8 (left) or 1013-4FA, made with protocol 2, (right) cells. n=3. (B) C-peptide and SIX2 immunostaining of stage 6 cells made with protocol 2 transduced with sh-ctrl or sh-SIX2-1 in the 1013-4FA background. (C) Dynamic glucose-stimulated insulin secretion of Stage 6 HUES8 cells transfected with control shRNA (sh-ctrl; n=4) or shRNA targeting SIX2 (sh-SIX2-2; n=4). Cells are perfused with 2 mM glucose except when indicated in a perifusion chamber. (D) Static glucose-stimulated insulin secretion of sh-ctrl or sh-SIX2-2 transduced Stage 6 HUES8 cells. n=5. (E) Dynamic glucose-stimulated insulin secretion of Stage 6 1013-4FA cells made with protocol 2 transfected with control shRNA (sh-ctrl; n=4) or shRNA targeting SIX2 (sh-SIX2-1; n=4). Cells are perfused with 2 mM glucose except when indicated in a perifusion chamber. (F) Static glucose-stimulated insulin secretion of sh-ctrl or sh-SIX2-1 transduced Stage 6 1013-4FA cells made with protocol 2. n=5. (G) CRISPR knock out strategy for the generation of the SIX2 KO HUES8 cell lines KO-SIX2-1 and KO-SIX2-2. HUES8 homozygous SIX2 KO clones were generated by deleting the SIX2 coding sequence using two gRNAs target flanking regions of the SIX2 coding sequence. Also shown are the primers used to validate deletion, “deletion primers” and “inside primers”. (H) PCR of SIX2 KO clones confirming SIX2 coding sequence deletion. “Deletion primers” will produce 3368 bp amplicon for wt but only ˜300 bp amplicon with successful deletion. “Inside primers” will produce ˜300 bp amplicon for wt but fail to amplify with successful deletion. (I) Next generation sequencing confirming deletion of SIX2 coding sequence in KO cell lines. The entire sequence between the 5′ and 3′ gRNA was absent (marked with red dash in reference). 1,085 total reads for KO-SIX2-1 clone and 840 total reads for KO-SIX2-2 clone were sequenced and 0% wt gRNA target sequence were detected and frame shifting indels totaled 100% for both clones. A few bp of SIX2 coding are leftover. (J) Real-time PCR measurements of SIX2 gene expression for Stage 6 cells made with protocol 2 from wt or KO SIX2 HUES8 backgrounds. n=5. (K) C-peptide and SIX2 immunostaining of Stage 6 cells made with protocol 2 from wt or KO SIX2 HUES8 backgrounds. Error bars represent s.e.m.

FIG. 6. Additional evaluation of SIX3 and SIX2. Related to FIG. 2. (A) Real-time PCR measurements of SIX3 in undifferentiated hESCs and at the end of each stage of the differentiation. Data is presented as the fold change relative to Stage 6 cells. All n=6, except PP2 n=3. (B) Real-time PCR measurements of SIX3 gene expression for Stage 6 cells transduced with sh-ctrl or sh-SIX2-1 (n=4; left) or SIX2 wt and KO cells (n=5; right). ns=p>0.05. (C) Immunostaining of NGN3 and NKX6-1 3 days into Stage 5. Scale bar=25 μm. (D) Flow cytometry plots of Stage 6 cells made with protocol 2 from the 1013-4FA background transduced with shRNA against GFP (control) and SIX2. (E) Flow cytometry plots of Stage 5 day 1 cells made with protocol 2 from the HUES8 background wt or KO for SIX2. n=4. ns by unpaired two-way t test. Error bars represent s.e.m.

FIG. 7. Additional RNA sequencing analysis. Related to FIG. 3. (A) Enriched gene sets for important β cell processes from the Molecular Signatures Database. (B) Additional enrichment plots. These are made with genes from the individual custom gene sets comprising 76 genes identified in Veres et al (Veres et al., 2019) and the top 424 genes identified in Nair et al (Nair et al., 2019) positively correlating with time and maturation in vitro. (C) Real-time PCR measurements of Stage 6 cells made with protocol 2 transduced with sh-ctrl or sh-SIX2-1 in the 1013-4FA background. *p<0.05, **p<0.01, ***p<0.001, ****p<0.0001 by two-way unpaired t test. Error bars represent s.e.m.

FIG. 8. Additional data on SIX2 KD and KO effects on SC-β cells. Related to FIG. 4. (A) Insulin content for Stage 6 cells transduced with sh-ctrl or sh-SIX2-1 in the 1013-4FA background. n=5. (B) Insulin content for Stage 6 cells wt or KO for SIX2. n=5. (C) OCR measurements for Stage 6 cells transduced with sh-ctrl or sh-SIX2-1 in the 1013-4FA background under basal conditions and after sequential injections of Oligomycin (OM), Carbonyl cyanide-4-(trifluoromethoxy)phenylhydrazone (FCCP), and Antimycin A with rotenone (AA/R). n=9 for sh-ctrl and n=10 for sh-SIX2-1. *p<0.05,***p<0.001 by two-way unpaired t test. Error bars represent s.e.m.

FIG. 9 is a graphical abstract of Example 1.

FIG. 10. SIX2 affects expression of stress genes. Relative gene expression of stress genes ATF4 and TXNIP.

FIG. 11. Regulators of SIX2. SC-β cells were treated with a library of compound to identify SIX2 regulators. (A) Positive regulators of SIX2 gene expression. (B) Negative Regulators of SIX2 gene expression. (C) Flow cytometry of SC-β cells treated with SIX2 regulators.

FIG. 12. SIX2 affects % of generated SC-β cells. Flow Cytometry of SC-β cells treated with SIX2 regulators for 12 days. It is presumed SC-β cells are C-Peptide+/NKX6.1+ cells.

FIG. 13. SIX2 regulators affect SC-β cell glucose stimulated insulin secretion.

FIG. 14. Glucose stimulated insulin secretion of single cell dispersed and plated down SC-β cells.

FIG. 15. Relative expression of SIX2 and SIX3 in human islets with Type 2 Diabetes (left) and relative expression of SIX2 in SC-β cells with exogenous stress (right). BFA=Brefeldin A, inhibits transport of proteins from ER to golgi.

FIG. 16. SIX2 KD reduces function in multiple cell types. (A) Reduced SIX2 expression with lentiviral knockdown. (B) Lower function, as measured by insulin secretion in response to glucose, with SIX2 KD in HUES8. (C) Lower function, as measured by insulin secretion in response to glucose, with SIX2 KD in alternated iPSC line, 1013.

FIG. 17. SIX2 KD increases expression of genes associated with oxidative stress. (A) Oxygen consumption rate for SIX2 KD. (B) Relative gene expression of stress genes ATF4 and TXNIP. CM=Cytokine Mix, ER inflammation; Tg=Thapsigargin, inhibits sarco/endocplasmic reticulum calcium ATPase; Tm=Tunicamycin, inhibits N-linked glycosylation, interrupts protein folding.

FIG. 18. Endoplasmic reticulum (ER) stress is increased with SIX2 KD in response to exogenous stress. (A) Increased ER stress gene expression. (B) Increased ER stress-mediated cell death. BFA=Brefeldin A, inhibits transport of proteins from ER to golgi; CM=Cytokine Mix, ER inflammation; Tg=Thapsigargin, inhibits sarco/endoplasmic reticulum calcium ATPase; Tm=Tunicamycin, inhibits N-linked glycosylation, interrupts protein folding.

### DETAILED DESCRIPTION OF THE INVENTION

The present disclosure is based, at least in part, on the discovery that the transcription factor, SIX2, drives stem-cell derived insulin-producing beta cell (SC-β cell) functional maturation. As described herein, SIX2 expression can be modulated to improve the differentiation efficiency of stem cells into mature SC-β cells capable of biphasic insulin secretion in response to glucose.

Although it was known that SIX2 is a marker of β cell maturation, it was not known that positively regulating this transcription factor would result in the discovered therapeutically beneficial features. The role of SIX2 in β cells is not well characterized, as it is not present in mouse β cells. These generated SC-β cells also allow for the study of SIX2 and its relation to function and stress.

As described herein, a 6-step stem cell differentiation protocol modified from Velazco-Cruz et al. 2019 (doi: 10.1016/j.stemcr.2018.12.012) (see also PCT/US2019/032643 (WU Ref No. T-018619), incorporated herein by reference) was used and the following specific features were observed: (1) SIX2 drives the acquisition of glucose-stimulated insulin secretion in SC-β cells; (2) SIX2 drives first and second phase insulin secretion; (3) SIX2 increases insulin gene expression and insulin content; (4) SIX2 augments glucose stimulated calcium flux; (5) SIX2 improves SC-β cell mitochondrial respiration; (6) SIX2 improves insulin secretion in response to beta cell secretagogues; and (7) SIX2 promotes maturation of SC-β cell transcriptome.

Further experiments determined that SIX2 is also associated with SC-β cell health. The following features were observed: (1) SIX2 knockdown reduces SC-β cell function; (2) knockdown of SIX2 in SC-β cells reduces mitochondrial respiration (basal oxygen consumption rate levels) and increases oxidative stress genes (TXNiP); (3) SIX2 KD SC-β cells have increased ER stress with the addition of exogenous stress compared to GFP control (increased ER stress gene expression, reduced β cell gene expression); (4) SIX2 KD SC-β are less resistant to ER-stress mediated cell death (increased Caspase 3/7 activation); (5) SIX2 decreases when ER stress is applied (displayed in Type 2 Diabetic compared to nondiabetic human islets, confirmed in SC-β cells with exogenous stress). It was also shown that SIX2 was reduced in diabetic human islets and SC-β cells with exogenous stress.

With current SC-β cell differentiation protocols, only a fraction of differentiated cells express SIX2 (e.g., SIX2(+) cells). As described herein, increasing SIX2 expression (e.g., increasing SIX2 expression on the beta cells and/or increasing the fraction of beta cells expressing SIX2) using positive SIX2 regulators can result in SIX2+ or CP+, or CP+/NKX6.1+ enriched populations or differentiated populations with increased SIX2 expression, function, or activity, and therapeutic applications.

As described herein, improving the differentiation of stem cells into insulin-producing pancreatic beta cells can be achieved by positively regulating SIX2 in SC-β cells.

Because the SC-β cells are generated by allowing EP cells to mature, the SIX2 positive regulator can be added directly to the EP cells to result in the SIX2-enhanced SC-β cells. As such, the SC-β cells that are used to generate the SIX2-enhanced SC-β cells can be replaced with endocrine progenitor (EP) cells or other β cell precursors or progenitors. For the purposes of this application and the methods described herein for generating SIX2-enhanced SC-β cells, the starting material of SC-β cells can be EP cells.

SIX2 Positive Regulators

As described herein, modulation of SIX2 (e.g., activation, upregulation, or positive regulation of expression, function, activity) in SC-β cells can be performed to enhance SC-β cell function and provide enhanced therapeutic benefit. As an example, SIX2 expression can be modulated by treating the SC-β cells with positive regulators (or positive modulators) of SIX2, such as a TGFβ agonist (e.g., TGFβ1 or TGFβ2), a glycogen synthase kinase 3 (GSK-3) inhibitor, a cocaine- and amphetamine-regulated transcript (CART) peptide fragment (e.g., CART 62-76 or CART 55-102), a FGFR inhibitor (e.g., AZD4547), or a p38 MAPK inhibitor (e.g., doramapimod). In some embodiments, the positive regulator of SIX2 activity or positive regulator SIX2 expression can be a DNA methyltransferase inhibitor (e.g., 5-Azacytidine (AZT)) or a SAHH and EZH2 inhibitor (e.g., 3-Deazaneplanocin A (DEZA)).

Agents described above can be those commercially available or known in the art (see e.g., Chang, Agonists and Antagonists of TGF-3 Family Ligands, Cold Spring Harb Perspect Biol. 2016 August; 8(8): a021923; Maqbool et al. GSK3 Inhibitors in the Therapeutic Development of Diabetes, Cancer and Neurodegeneration: Past, Present and Future, Curr Pharm Des. 2017 Nov. 16; 23(29):4332-4350; Eldar-Finkelman et al. GSK-3 inhibitors: preclinical and clinical focus on CNS Front. Mol. Neurosci., 31 Oct. 2011; Rogge et al., CART peptides: regulators of body weight, reward and other functions, Nat Rev Neurosci. Nat Rev Neurosci. 2008 October; 9(10): 747-758; Dai et al. Fibroblast Growth Factor Receptors (FGFRs): Structures and Small Molecule Inhibitors, Cells. 2019 June; 8(6): 614; Lee et al., Recent Advances in the Inhibition of p38 MAPK as a Potential Strategy for the Treatment of Alzheimer's Disease, Molecules. 2017 August; 22(8): 1287; Gnyszka et al., DNA methyltransferase inhibitors and their emerging role in epigenetic therapy of cancer, Anticancer Res. 2013 August; 33(8):2989-96). Other SAHH inhibitors are also commercially available, such as D-Eritadenine, Adenosine, 3-Deazaadenosine, Tubercidin, or 3-Deazaneplanocin. Commercially available EZH2 inhibitors can include, for example, UNC 1999, UNC 2399, JQEZ5, 3-Deazaneplanocin A hydrochloride, or PF 06726304 acetate.

The population of SC-β cells to be treated with the SIX2 positive regulator can be any number of SC-β cells. For example, the population of SC-β cells can be between about 10⁰ cells/mL and about 10¹⁰ cells/mL. As another example, the population of SC-β cells can be about 10⁰ cells/mL, 10¹ cells/mL, 10² cells/mL, 10³ cells/mL, 10⁴ cells/mL, 10⁵ cells/mL, 10⁶ cells/mL, 10⁷ cells/mL, 10⁶ cells/mL, 10⁹ cells/mL, or about 10¹⁰ cells/mL.

The amount of a SIX2 positive regulator sufficient to form SIX2-enhanced SC-β cells can be any amount and concentration sufficient to form SIX2-enhanced SC-β cells. This amount or concentration can be dependent on the type of activator used (e.g., small molecule or growth factor). For example, a SIX2 positive regulator comprising a growth factor (e.g., TGFb) can be used at a concentration between about 1 pg/mL and about 1 mg/mL. As another example, a SIX2 positive regulator comprising a small molecule (e.g., CART 62-76) can be used at a concentration between about 1 nM and about 100 μM.

As an example, a SIX2 positive regulator can be used in a concentration of about 1 pg/mL; about 10 pg/mL; about 20 pg/mL; about 30 pg/mL; about 40 pg/mL; about 50 pg/mL; about 60 pg/mL; about 70 pg/mL; about 80 pg/mL; about 90 pg/mL; about 100 pg/mL; about 110 pg/mL; about 120 pg/mL; about 130 pg/mL; about 140 pg/mL; about 150 pg/mL; about 160 pg/mL; about 170 pg/mL; about 180 pg/mL; about 190 pg/mL; about 200 pg/mL; about 210 pg/mL; about 220 pg/mL; about 230 pg/mL; about 240 pg/mL; about 250 pg/mL; about 260 pg/mL; about 270 pg/mL; about 280 pg/mL; about 290 pg/mL; about 300 pg/mL; about 310 pg/mL; about 320 pg/mL; about 330 pg/mL; about 340 pg/mL; about 350 pg/mL; about 360 pg/mL; about 370 pg/mL; about 380 pg/mL; about 390 pg/mL; about 400 pg/mL; about 410 pg/mL; about 420 pg/mL; about 430 pg/mL; about 440 pg/mL; about 450 pg/mL; about 460 pg/mL; about 470 pg/mL; about 480 pg/mL; about 490 pg/mL; about 500 pg/mL; about 510 pg/mL; about 520 pg/mL; about 530 pg/mL; about 540 pg/mL; about 550 pg/mL; about 560 pg/mL; about 570 pg/mL; about 580 pg/mL; about 590 pg/mL; about 600 pg/mL; about 610 pg/mL; about 620 pg/mL; about 630 pg/mL; about 640 pg/mL; about 650 pg/mL; about 660 pg/mL; about 670 pg/mL; about 680 pg/mL; about 690 pg/mL; about 700 pg/mL; about 710 pg/mL; about 720 pg/mL; about 730 pg/mL; about 740 pg/mL; about 750 pg/mL; about 760 pg/mL; about 770 pg/mL; about 780 pg/mL; about 790 pg/mL; about 800 pg/mL; about 810 pg/mL; about 820 pg/mL; about 830 pg/mL; about 840 pg/mL; about 850 pg/mL; about 860 pg/mL; about 870 pg/mL; about 880 pg/mL; about 890 pg/mL; about 900 pg/mL; about 910 pg/mL; about 920 pg/mL; about 930 pg/mL; about 940 pg/mL; about 950 pg/mL; about 960 pg/mL; about 970 pg/mL; about 980 pg/mL; about 990 pg/mL; about 1 ng/mL; about 10 ng/mL; about 20 ng/mL; about 30 ng/mL; about 40 ng/mL; about 50 ng/mL; about 60 ng/mL; about 70 ng/mL; about 80 ng/mL; about 90 ng/mL; about 100 ng/mL; about 110 ng/mL; about 120 ng/mL; about 130 ng/mL; about 140 ng/mL; about 150 ng/mL; about 160 ng/mL; about 170 ng/mL; about 180 ng/mL; about 190 ng/mL; about 200 ng/mL; about 210 ng/mL; about 220 ng/mL; about 230 ng/mL; about 240 ng/mL; about 250 ng/mL; about 260 ng/mL; about 270 ng/mL; about 280 ng/mL; about 290 ng/mL; about 300 ng/mL; about 310 ng/mL; about 320 ng/mL; about 330 ng/mL; about 340 ng/mL; about 350 ng/mL; about 360 ng/mL; about 370 ng/mL; about 380 ng/mL; about 390 ng/mL; about 400 ng/mL; about 410 ng/mL; about 420 ng/mL; about 430 ng/mL; about 440 ng/mL; about 450 ng/mL; about 460 ng/mL; about 470 ng/mL; about 480 ng/mL; about 490 ng/mL; about 500 ng/mL; about 510 ng/mL; about 520 ng/mL; about 530 ng/mL; about 540 ng/mL; about 550 ng/mL; about 560 ng/mL; about 570 ng/mL; about 580 ng/mL; about 590 ng/mL; about 600 ng/mL; about 610 ng/mL; about 620 ng/mL; about 630 ng/mL; about 640 ng/mL; about 650 ng/mL; about 660 ng/mL; about 670 ng/mL; about 680 ng/mL; about 690 ng/mL; about 700 ng/mL; about 710 ng/mL; about 720 ng/mL; about 730 ng/mL; about 740 ng/mL; about 750 ng/mL; about 760 ng/mL; about 770 ng/mL; about 780 ng/mL; about 790 ng/mL; about 800 ng/mL; about 810 ng/mL; about 820 ng/mL; about 830 ng/mL; about 840 ng/mL; about 850 ng/mL; about 860 ng/mL; about 870 ng/mL; about 880 ng/mL; about 890 ng/mL; about 900 ng/mL; about 910 ng/mL; about 920 ng/mL; about 930 ng/mL; about 940 ng/mL; about 950 ng/mL; about 960 ng/mL; about 970 ng/mL; about 980 ng/mL; about 990 ng/mL; or about 1000 ng/mL; about 1 pg/mL; about 10 pg/mL; about 20 pg/mL; about 30 pg/mL; about 40 pg/mL; about 50 pg/mL; about 60 pg/mL; about 70 pg/mL; about 80 pg/mL; about 90 pg/mL; about 100 pg/mL; about 110 pg/mL; about 120 pg/mL; about 130 pg/mL; about 140 pg/mL; about 150 pg/mL; about 160 pg/mL; about 170 pg/mL; about 180 pg/mL; about 190 pg/mL; about 200 pg/mL; about 210 pg/mL; about 220 pg/mL; about 230 pg/mL; about 240 pg/mL; about 250 pg/mL; about 260 pg/mL; about 270 pg/mL; about 280 pg/mL; about 290 pg/mL; about 300 pg/mL; about 310 pg/mL; about 320 pg/mL; about 330 pg/mL; about 340 pg/mL; about 350 pg/mL; about 360 pg/mL; about 370 pg/mL; about 380 pg/mL; about 390 pg/mL; about 400 pg/mL; about 410 pg/mL; about 420 pg/mL; about 430 pg/mL; about 440 pg/mL; about 450 pg/mL; about 460 pg/mL; about 470 pg/mL; about 480 pg/mL; about 490 pg/mL; about 500 pg/mL; about 510 pg/mL; about 520 pg/mL; about 530 pg/mL; about 540 pg/mL; about 550 pg/mL; about 560 pg/mL; about 570 pg/mL; about 580 pg/mL; about 590 pg/mL; about 600 pg/mL; about 610 pg/mL; about 620 pg/mL; about 630 pg/mL; about 640 pg/mL; about 650 pg/mL; about 660 pg/mL; about 670 pg/mL; about 680 pg/mL; about 690 pg/mL; about 700 pg/mL; about 710 pg/mL; about 720 pg/mL; about 730 pg/mL; about 740 pg/mL; about 750 pg/mL; about 760 pg/mL; about 770 pg/mL; about 780 pg/mL; about 790 pg/mL; about 800 pg/mL; about 810 pg/mL; about 820 pg/mL; about 830 pg/mL; about 840 pg/mL; about 850 pg/mL; about 860 pg/mL; about 870 pg/mL; about 880 pg/mL; about 890 pg/mL; about 900 pg/mL; about 910 pg/mL; about 920 pg/mL; about 930 pg/mL; about 940 pg/mL; about 950 pg/mL; about 960 pg/mL; about 970 pg/mL; about 980 pg/mL; about 990 pg/mL; about 1 mg/mL; about 10 mg/mL; about 20 mg/mL; about 30 mg/mL; about 40 mg/mL; about 50 mg/mL; about 60 mg/mL; about 70 mg/mL; about 80 mg/mL; about 90 mg/mL; about 100 mg/mL; about 110 mg/mL; about 120 mg/mL; about 130 mg/mL; about 140 mg/mL; about 150 mg/mL; about 160 mg/mL; about 170 mg/mL; about 180 mg/mL; about 190 mg/mL; about 200 mg/mL; about 210 mg/mL; about 220 mg/mL; about 230 mg/mL; about 240 mg/mL; about 250 mg/mL; about 260 mg/mL; about 270 mg/mL; about 280 mg/mL; about 290 mg/mL; about 300 mg/mL; about 310 mg/mL; about 320 mg/mL; about 330 mg/mL; about 340 mg/mL; about 350 mg/mL; about 360 mg/mL; about 370 mg/mL; about 380 mg/mL; about 390 mg/mL; about 400 mg/mL; about 410 mg/mL; about 420 mg/mL; about 430 mg/mL; about 440 mg/mL; about 450 mg/mL; about 460 mg/mL; about 470 mg/mL; about 480 mg/mL; about 490 mg/mL; about 500 mg/mL; about 510 mg/mL; about 520 mg/mL; about 530 mg/mL; about 540 mg/mL; about 550 mg/mL; about 560 mg/mL; about 570 mg/mL; about 580 mg/mL; about 590 mg/mL; about 600 mg/mL; about 610 mg/mL; about 620 mg/mL; about 630 mg/mL; about 640 mg/mL; about 650 mg/mL; about 660 mg/mL; about 670 mg/mL; about 680 mg/mL; about 690 mg/mL; about 700 mg/mL; about 710 mg/mL; about 720 mg/mL; about 730 mg/mL; about 740 mg/mL; about 750 mg/mL; about 760 mg/mL; about 770 mg/mL; about 780 mg/mL; about 790 mg/mL; about 800 mg/mL; about 810 mg/mL; about 820 mg/mL; about 830 mg/mL; about 840 mg/mL; about 850 mg/mL; about 860 mg/mL; about 870 mg/mL; about 880 mg/mL; about 890 mg/mL; about 900 mg/mL; about 910 mg/mL; about 920 mg/mL; about 930 mg/mL; about 940 mg/mL; about 950 mg/mL; about 960 mg/mL; about 970 mg/mL; about 980 mg/mL; about 990 mg/mL; or about 1 g/mL.

As another example, a SIX2 positive regulator can be used in a concentration of about 1 pM; about 10 pM; about 20 pM; about 30 pM; about 40 pM; about 50 pM; about 60 pM; about 70 pM; about 80 pM; about 90 pM; about 100 pM; about 110 pM; about 120 pM; about 130 pM; about 140 pM; about 150 pM; about 160 pM; about 170 pM; about 180 pM; about 190 pM; about 200 pM; about 210 pM; about 220 pM; about 230 pM; about 240 pM; about 250 pM; about 260 pM; about 270 pM; about 280 pM; about 290 pM; about 300 pM; about 310 pM; about 320 pM; about 330 pM; about 340 pM; about 350 pM; about 360 pM; about 370 pM; about 380 pM; about 390 pM; about 400 pM; about 410 pM; about 420 pM; about 430 pM; about 440 pM; about 450 pM; about 460 pM; about 470 pM; about 480 pM; about 490 pM; about 500 pM; about 510 pM; about 520 pM; about 530 pM; about 540 pM; about 550 pM; about 560 pM; about 570 pM; about 580 pM; about 590 pM; about 600 pM; about 610 pM; about 620 pM; about 630 pM; about 640 pM; about 650 pM; about 660 pM; about 670 pM; about 680 pM; about 690 pM; about 700 pM; about 710 pM; about 720 pM; about 730 pM; about 740 pM; about 750 pM; about 760 pM; about 770 pM; about 780 pM; about 790 pM; about 800 pM; about 810 pM; about 820 pM; about 830 pM; about 840 pM; about 850 pM; about 860 pM; about 870 pM; about 880 pM; about 890 pM; about 900 pM; about 910 pM; about 920 pM; about 930 pM; about 940 pM; about 950 pM; about 960 pM; about 970 pM; about 980 pM; about 990 pM; about 1 nM; about 10 nM; about 20 nM; about 30 nM; about 40 nM; about 50 nM; about 60 nM; about 70 nM; about 80 nM; about 90 nM; about 100 nM; about 110 nM; about 120 nM; about 130 nM; about 140 nM; about 150 nM; about 160 nM; about 170 nM; about 180 nM; about 190 nM; about 200 nM; about 210 nM; about 220 nM; about 230 nM; about 240 nM; about 250 nM; about 260 nM; about 270 nM; about 280 nM; about 290 nM; about 300 nM; about 310 nM; about 320 nM; about 330 nM; about 340 nM; about 350 nM; about 360 nM; about 370 nM; about 380 nM; about 390 nM; about 400 nM; about 410 nM; about 420 nM; about 430 nM; about 440 nM; about 450 nM; about 460 nM; about 470 nM; about 480 nM; about 490 nM; about 500 nM; about 510 nM; about 520 nM; about 530 nM; about 540 nM; about 550 nM; about 560 nM; about 570 nM; about 580 nM; about 590 nM; about 600 nM; about 610 nM; about 620 nM; about 630 nM; about 640 nM; about 650 nM; about 660 nM; about 670 nM; about 680 nM; about 690 nM; about 700 nM; about 710 nM; about 720 nM; about 730 nM; about 740 nM; about 750 nM; about 760 nM; about 770 nM; about 780 nM; about 790 nM; about 800 nM; about 810 nM; about 820 nM; about 830 nM; about 840 nM; about 850 nM; about 860 nM; about 870 nM; about 880 nM; about 890 nM; about 900 nM; about 910 nM; about 920 nM; about 930 nM; about 940 nM; about 950 nM; about 960 nM; about 970 nM; about 980 nM; about 990 nM; or about 1000 nM; about 1 μM; about 10 μM; about 20 μM; about 30 μM; about 40 μM; about 50 μM; about 60 μM; about 70 μM; about 80 μM; about 90 μM; about 100 μM; about 110 μM; about 120 μM; about 130 μM; about 140 μM; about 150 μM; about 160 μM; about 170 μM; about 180 μM; about 190 μM; about 200 μM; about 210 μM; about 220 μM; about 230 μM; about 240 μM; about 250 μM; about 260 μM; about 270 μM; about 280 μM; about 290 μM; about 300 μM; about 310 μM; about 320 μM; about 330 μM; about 340 μM; about 350 μM; about 360 μM; about 370 μM; about 380 μM; about 390 μM; about 400 μM; about 410 μM; about 420 μM; about 430 μM; about 440 μM; about 450 μM; about 460 μM; about 470 μM; about 480 μM; about 490 μM; about 500 μM; about 510 μM; about 520 μM; about 530 μM; about 540 μM; about 550 μM; about 560 μM; about 570 μM; about 580 μM; about 590 μM; about 600 μM; about 610 μM; about 620 μM; about 630 μM; about 640 μM; about 650 μM; about 660 μM; about 670 μM; about 680 μM; about 690 μM; about 700 μM; about 710 μM; about 720 μM; about 730 μM; about 740 μM; about 750 μM; about 760 μM; about 770 μM; about 780 μM; about 790 μM; about 800 μM; about 810 μM; about 820 μM; about 830 μM; about 840 μM; about 850 μM; about 860 μM; about 870 μM; about 880 μM; about 890 μM; about 900 μM; about 910 μM; about 920 μM; about 930 μM; about 940 μM; about 950 μM; about 960 μM; about 970 μM; about 980 μM; about 990 μM; about 1 mM; about 10 mM; about 20 mM; about 30 mM; about 40 mM; about 50 mM; about 60 mM; about 70 mM; about 80 mM; about 90 mM; about 100 mM; about 110 mM; about 120 mM; about 130 mM; about 140 mM; about 150 mM; about 160 mM; about 170 mM; about 180 mM; about 190 mM; about 200 mM; about 210 mM; about 220 mM; about 230 mM; about 240 mM; about 250 mM; about 260 mM; about 270 mM; about 280 mM; about 290 mM; about 300 mM; about 310 mM; about 320 mM; about 330 mM; about 340 mM; about 350 mM; about 360 mM; about 370 mM; about 380 mM; about 390 mM; about 400 mM; about 410 mM; about 420 mM; about 430 mM; about 440 mM; about 450 mM; about 460 mM; about 470 mM; about 480 mM; about 490 mM; about 500 mM; about 510 mM; about 520 mM; about 530 mM; about 540 mM; about 550 mM; about 560 mM; about 570 mM; about 580 mM; about 590 mM; about 600 mM; about 610 mM; about 620 mM; about 630 mM; about 640 mM; about 650 mM; about 660 mM; about 670 mM; about 680 mM; about 690 mM; about 700 mM; about 710 mM; about 720 mM; about 730 mM; about 740 mM; about 750 mM; about 760 mM; about 770 mM; about 780 mM; about 790 mM; about 800 mM; about 810 mM; about 820 mM; about 830 mM; about 840 mM; about 850 mM; about 860 mM; about 870 mM; about 880 mM about 890 mM; about 900 mM; about 910 mM; about 920 mM; about 930 mM; about 940 mM; about 950 mM; about 960 mM; about 970 mM; about 980 mM about 990 mM; or about 1 M.

The amount of SIX2 positive regulator is in an amount sufficient to increase the % of CP+/NKX6.1+ cells to more than about 25% of the total population of cells to at least about 60%. The amount of SIX2 positive regulator is an amount sufficient to increase SIX2 expression, function, or activity by at least about 5% to at least about 50% compared to an SC-β cell not treated with the SIX2 positive regulator. The amount of SIX2 positive regulator is in an amount sufficient to increase the % of CP+/NKX6.1+ cells to more than an SC-β cell not treated with the SIX2 positive regulator. For example, CP+, NKX6.1+, or SIX2+ cells can be increased by at least about 5% up to about 50%. As an example, CP+ cells can be increased by about 1%; about 2%; about 3%; about 4%; about 5%; about 6%; about 7%; about 8%; about 9%; about 10%; about 11%; about 12%; about 13%; about 14%; about 15%; about 16%; about 17%; about 18%; about 19%; about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; about 40%; about 41%; about 42%; about 43%; about 44%; about 45%; about 46%; about 47%; about 48%; about 49%; about 50%; about 51%; about 52%; about 53%; about 54%; about 55%; about 56%; about 57%; about 58%; about 59%; about 60%; about 61%; about 62%; about 63%; about 64%; about 65%; about 66%; about 67%; about 68%; about 69%; about 70%; about 71%; about 72%; about 73%; about 74%; about 75%; about 76%; about 77%; about 78%; about 79%; about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; about 99%; about 100%; about 101%; about 102%; about 103%; about 104%; about 105%; about 106%; about 107%; about 108%; about 109%; about 110%; about 111%; about 112%; about 113%; about 114%; about 115%; about 116%; about 117%; about 118%; about 119%; about 120%; about 121%; about 122%; about 123%; about 124%; about 125%; about 126%; about 127%; about 128%; about 129%; about 130%; about 131%; about 132%; about 133%; about 134%; about 135%; about 136%; about 137%; about 138%; about 139%; about 140%; about 141%; about 142%; about 143%; about 144%; about 145%; about 146%; about 147%; about 148%; about 149%; about 150%; about 151%; about 152%; about 153%; about 154%; about 155%; about 156%; about 157%; about 158%; about 159%; about 160%; about 161%; about 162%; about 163%; about 164%; about 165%; about 166%; about 167%; about 168%; about 169%; about 170%; about 171%; about 172%; about 173%; about 174%; about 175%; about 176%; about 177%; about 178%; about 179%; about 180%; about 181%; about 182%; about 183%; about 184%; about 185%; about 186%; about 187%; about 188%; about 189%; about 190%; about 191%; about 192%; about 193%; about 194%; about 195%; about 196%; about 197%; about 198%; about 199%; about 200%; about 201%; about 102%; about 203%; about 204%; about 205%; about 206%; about 207%; about 208%; about 209%; about 210%; about 211%; about 212%; about 213%; about 214%; about 215%; about 216%; about 217%; about 218%; about 219%; about 220%; about 221%; about 222%; about 223%; about 224%; about 225%; about 226%; about 227%; about 228%; about 229%; about 230%; about 231%; about 232%; about 233%; about 234%; about 235%; about 236%; about 237%; about 238%; about 239%; about 240%; about 241%; about 242%; about 243%; about 244%; about 245%; about 246%; about 247%; about 248%; about 249%; about 250%; about 251%; about 252%; about 253%; about 254%; about 255%; about 256%; about 257%; about 258%; about 259%; about 260%; about 261%; about 262%; about 263%; about 264%; about 265%; about 266%; about 267%; about 268%; about 269%; about 270%; about 271%; about 272%; about 273%; about 274%; about 275%; about 276%; about 277%; about 278%; about 279%; about 280%; about 281%; about 282%; about 283%; about 284%; about 285%; about 286%; about 287%; about 288%; about 289%; about 290%; about 291%; about 292%; about 293%; about 294%; about 295%; about 296%; about 297%; about 298%; about 299%; or about 300%.

The amount of time a SIX2 positive regulator is incubated with (in fluid contact with) the SC-β cells is at least about 6 hours, about 12 hours, about 1 day, at least about 2 days, or between about 1 day and 2 weeks. For example, the SIX2 positive regulator can be incubated with the SC-β cells for about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours (about 1 day); about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; about 48 hours (about 2 days); about 3 days; about 4 days; 5 days; about 6 days; about 7 days; about 8 days; about 9 days; about 10 days; about 11 days; about 12 days; about 13 days; about 14 days; about 15 days; about 16 days; about 17 days; about 18 days; about 19 days; about 20 days; about 21 days; about 22 days; about 23 days; about 24 days; about 25 days; about 26 days; about 27 days; about 28 days; about 29 days; or about 30 days.

Single Cell Dispersion and Plating

As described herein, another modification to the protocol is the single cell dispersion and plating of SC-β cells, rather than conventional SC-β cell clusters. Surprisingly, the single SC-β cells are capable of secreting insulin in response to glucose. The single cell plating modification allows the protocol described herein to be amenable to high-throughput screening with SC-β cells. This finding is significant in that it can facilitate high-throughput screens using SC-β cells. For example, the SC-β cells generated by the protocol described herein can be used in drug discovery screens.

SC-Beta Cell Therapy

SC-β cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. Here, transplanting SC-beta cells can be used to treat diabetes.

Stem cell and cell transplantation have gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogenic strategy is that unmatched allogeneic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans.

Xenogeneic cell therapies can involve human cell transplantation into experimental animal models for assessment of efficacy and safety or enable xenogeneic strategies to humans as well.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, subcutaneous, intranasal, epidural, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating or preventing diabetes a subject in need of administration of a therapeutically effective amount of stem-cells derived functionally mature beta cells so as to induce insulin secretion. Also provided is a process of using the generated cells for cell replacement therapies or stem cell transplant. For example, the disclosed compositions and methods can be used to treat diabetes or other disease associated with dysfunctional endodermal cells in a subject in need of administration of a therapeutically effective amount of cells of endodermal lineage or beta cells, so as to induce insulin secretion. Activation of SIX2 can be performed on SC-beta cells, endogenous beta cells, native beta cells, donor beta cells, or transplanted beta cells (auto- or allo-transplanted).

Methods described herein are generally performed on a subject in need thereof. A subject in need of the therapeutic methods described herein can be a subject having, diagnosed with, suspected of having, or at risk for developing diabetes. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of functionally mature beta cells is, for example, that amount that would cause the desired therapeutic effect in a subject while minimizing undesired side effects. In various embodiments, an effective amount of functionally mature beta cells described herein can substantially inhibit diabetes, slow the progression of diabetes, or limit the development of diabetes.

According to the methods described herein, administration can be implantation or transplantation as well as parenteral, intradermal, intramuscular, intraperitoneal, intravenous, or subcutaneous.

When used in the treatments described herein, a therapeutically effective amount of functionally mature beta cells can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat diabetes.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of functionally mature beta cells can occur as a single event or over a time course of treatment. For example, functionally mature beta cells can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for diabetes.

Functionally mature beta cells can be administered simultaneously or sequentially with another agent, such as an antibiotic, an anti-inflammatory, or another agent. For example, functionally mature beta cells can be administered simultaneously with another agent, such as an antibiotic or an anti-inflammatory. Simultaneous administration can occur through administration of separate compositions, each containing one or more functionally mature beta cells, an antibiotic, an anti-inflammatory, or another agent. Simultaneous administration can occur through administration of one composition containing two or more functionally mature beta cells, an antibiotic, an anti-inflammatory, or another agent. Functionally mature beta cells can be administered sequentially with an antibiotic, an anti-inflammatory, or another agent. For example, functionally mature beta cells can be administered before or after administration of an antibiotic, an anti-inflammatory, or another agent.

Administration

Agents and compositions described herein can be administered according to methods described herein in a variety of means known to the art.

As discussed above, administration can be implantation, transplantation, parenteral, pulmonary, intradermal, intramuscular, intraperitoneal, intravenous, or subcutaneous.

Agents and compositions described herein can be administered in a variety of methods well known in the arts. Administration can include, for example, methods involving implantation of cells secreting the factor of interest, biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Screening

Also provided are methods for screening for therapeutic compounds. The screening method can comprise providing a cell (e.g., SIX2-enhanced SC-β cell) generated by any of the methods described herein and introducing a compound or composition (e.g., a secretagogue) to the cell. For example, the screening method can be used for drug screening or toxicity screening on any cell of endodermal lineage or beta cell provided herein.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to SC-β cells, stem cells, or precursors or progenitors thereof, media, and agents as described herein such as a positive regulator of SIX2 activity or SIX2 expression (e.g., a TGFβ agonist such as TGFβ1 or TGFβ2; a GSK-3 inhibitor such as CHIR99021; a CART peptide fragment such as CART 62-76 or CART 55-102; a FGFR inhibitor such as AZD4547; or a p38 MAPK inhibitor such as doramapimod), serum free media, TGFβ/Activin agonist or a glycogen synthase kinase 3 (GSK) inhibitor or WNT agonist, a FGFR2b agonist, an RAR agonist, and optionally a rho kinase inhibitor, a smoothened antagonist, a FGFR2b agonist, a protein kinase C activator, or a BMP type 1 receptor inhibitor, an Alk5 inhibitor, a gamma secretase inhibitor, SANT1, Erbb1 (EGFR) or Erbb4 agonist. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline, each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: SIX2 Regulates Human B Cell Differentiation from Stem Cells and Functional Maturation In Vitro

The following example describes how the functional maturation of stem-cell derived beta cells (SC-β cells) is regulated by the transcription factor SIX2. Here, it is shown that SIX2 regulates the functional maturation of stem cell-derived p cells; knockdown and knockout of SIX2 impairs static and dynamic GSIS; SIX2 regulates in vitro β cell gene expression signature; and SIX2 expression is heterogeneous within the SC-β cell population, published as Velazco-Cruz et al., SIX2 Regulates Human β Cell Differentiation from Stem Cells and Functional Maturation In Vitro, Cell Reports, Volume 31, Issue 8, 2020, 107687, incorporated herein by reference.

Human pluripotent stem cells can be induced to differentiate into insulin-producing pancreatic beta cells (SC-β cells) applications.

Here, it was discovered that expression of transcription factor SIX2 drives maturation of SC-β cells and SIX2 expression can be modulated to improve differentiation efficiency.

As such, contacting the beta cell with a positive regulator of SIX2 activity or SIX2 expression results in the beta cell having enhanced glucose-stimulated insulin secretion.

### Summary

The generation of insulin-secreting β cells in vitro is a promising approach for diabetes cell therapy. Human embryonic stem cells (hESCs) and human induced pluripotent stem cells (hiPSCs) are differentiated to β cells (SC-β cells) and mature to undergo glucose-stimulated insulin secretion, but molecular regulation of this defining β cell phenotype is unknown. Here, it was shown that the maturation of SC-β cells is regulated by the transcription factor SIX2. Knockdown (KD) or knockout (KO) of SIX2 in SC-β cells drastically limits glucose-stimulated insulin secretion in both static and dynamic assays, along with the upstream processes of cytoplasmic calcium flux and mitochondrial respiration. Furthermore, SIX2 regulates the expression of genes associated with these key β cell processes, and its expression is restricted to endocrine cells. These results demonstrate that expression of SIX2 influences the generation of human SC-β cells in vitro.

### Introduction

Pancreatic β cells regulate blood glucose levels by secreting a precise amount of insulin in response to changes in extracellular glucose, and death or dysfunction of these cells results in diabetes. Transplantation of insulin-secreting cells shows promise to be an effective treatment for diabetes (Bellin et al., 2012, McCall and Shapiro, 2012, Millman and Pagliuca, 2017), and a small number of patients who have received such implants from cadaveric donors remain normoglycemic for years. Scarcity and high variability of donor islets limit this approach, however (McCall and Shapiro, 2012).

To overcome this limitation, strategies for specifying β cells from human embryonic stem cells (hESCs) in vitro have been described recently (Pagliuca et al., 2014, Rezania et al., 2014). These approaches use growth factors and small molecules to mimic native β cell development by first specifying definitive endoderm (D'Amour et al., 2005), followed by the generation of NKX6-1⁺ pancreatic progenitors (D'Amour et al., 2006). These progenitors are specified into endocrine via the expression of NEUROG3 (NGN3) (Gu et al., 2002) and are subsequently matured into SC-β cells and other islet endocrine cell types (Veres et al., 2019). More recent studies have defined conditions that greatly improve the functional maturation of SC-β cells, achieving first- and second-phase insulin secretion (Hogrebe et al., 2020, Velazco-Cruz et al., 2019). While a large number of genes that temporally correlate with maturation have been identified (Nair et al., 2019, Veres et al., 2019), the molecular mechanisms controlling this functional maturation are unclear, hampering further improvements in function.

To investigate the functional maturation of human β cells in vitro, the homeobox transcription factor SIX2 was studied during differentiation to SC-β cells. SIX2 is not expressed in rodent β cells (Segerstolpe et al., 2016, Xin et al., 2016), which limits the use of conventional approaches for its study, including commonly used insulinoma cell lines and animal models. SIX2 has recently been identified as being expressed in human β cells and linked to type 2 diabetes and aging (Arda et al., 2016, Hachiya et al., 2017, Kim et al., 2011, Spracklen et al., 2018).

Here, it is reported that SIX2 is key for generating functional SC-β cells in vitro. Using short hairpin RNA (shRNA) and CRISPR-Cas9 to knock down (KD) SIX2 expression or knock out (KO) the SIX2 gene, respectively, it was shown that both static and dynamic glucose-stimulated insulin secretion are severely hampered with reduced SIX2 expression. Upstream processes of cytoplasmic calcium flux and mitochondrial respiration are similarly reduced. Using RNA sequencing, a large number of genes associated with maturation and β cell function were observed to be reduced with the KD of SIX2, including gene sets associated temporally with SC-β cell maturation in vitro from other research groups.

Results

SIX2 is Crucial for Acquisition of Glucose-Stimulated Insulin Secretion

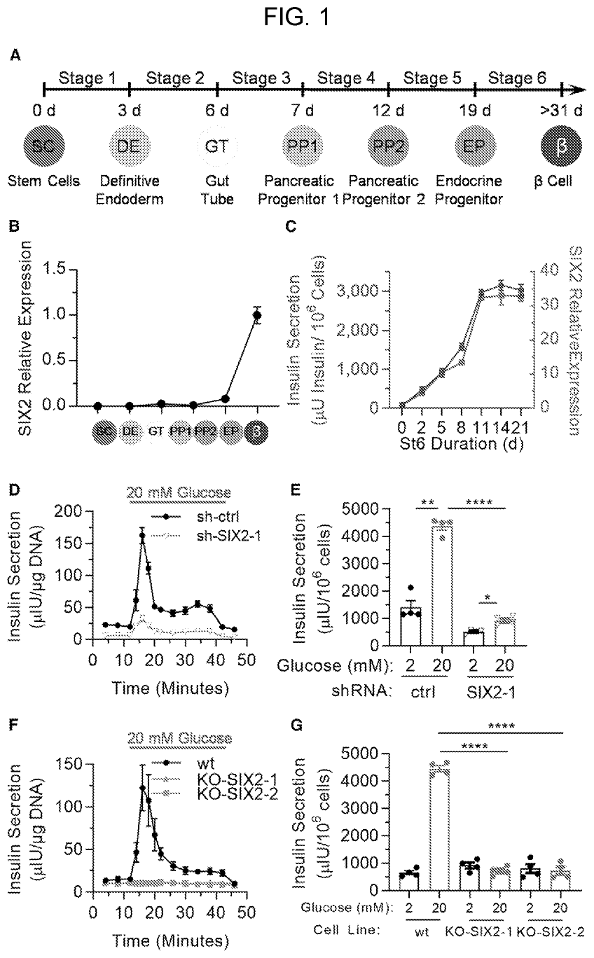

Because SIX2 is expressed in human β cells, but its regulatory role during β cell differentiation and maturation is uncharacterized, its gene expression during this 6-stage differentiation protocol was measured (FIG. 1A). A notably large increase in expression during the maturation of endocrine progenitors to SC-β cells were observed a (FIG. 1B). Closer inspection of stage 6 revealed that the gene expression of SIX2 increased 32.5±0.9 times during the first 11 days, correlating with increases in insulin protein secretion per cell for the same time period (FIG. 1C).

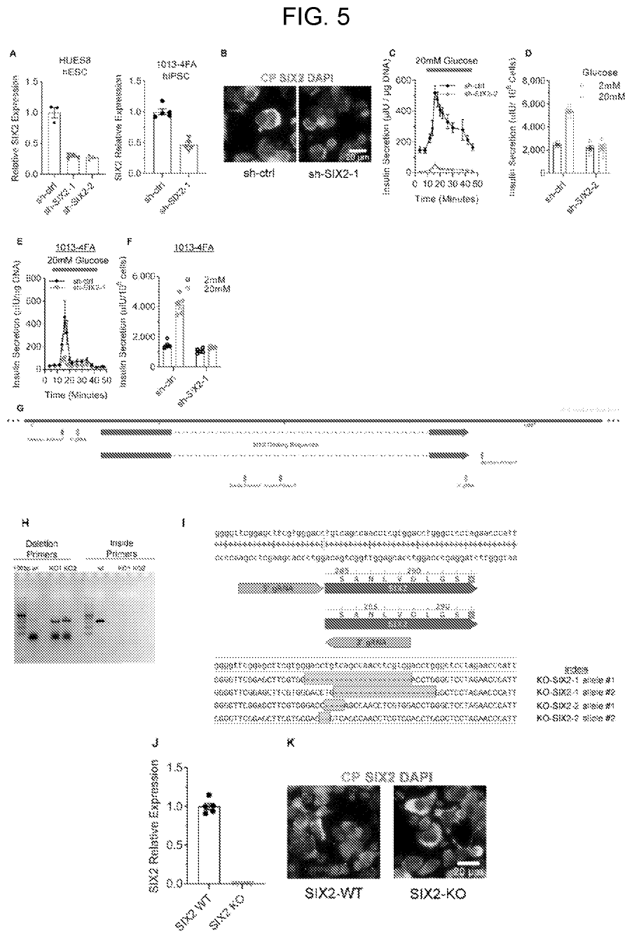

To further study SIX2 using this SC-β cell platform, 2 lentiviruses carrying shRNAs (sh-SIX2-1 and sh-SIX2-2) to KD the expression of SIX2 were generated (FIG. 5A and FIG. 5B). For these KD studies, cells were transduced on the first day of stage 6 to limit the emergence of SIX2 expression with time. KD of SIX2 in differentiating hESCs (HUES8) and human induced pluripotent stem cells (hiPSCs) (1013-4FA) lines resulted in significant reductions in both dynamic and static glucose-stimulated insulin secretion assays (FIG. 1D, FIG. 1E, and FIG. 5C-FIG. 5F). While peaks in the dynamic insulin secretion profile resembling first- and second-phase insulin secretion were still observed, the overall amount of insulin secreted per DNA was 4.2±1.2 times lower at high glucose with SIX2 KD (FIG. 1D). Similarly, for the static assay, while the cells still responded, albeit more weakly, to glucose by secreting elevated insulin, insulin secretion per cell was 4.7±0.8 times lower at high glucose with SIX2 KD (FIG. 1E). It is noted that the magnitude of the relative insulin secretion at low glucose differed between static and dynamic assays, perhaps due to fluid shear or paracrine effects.

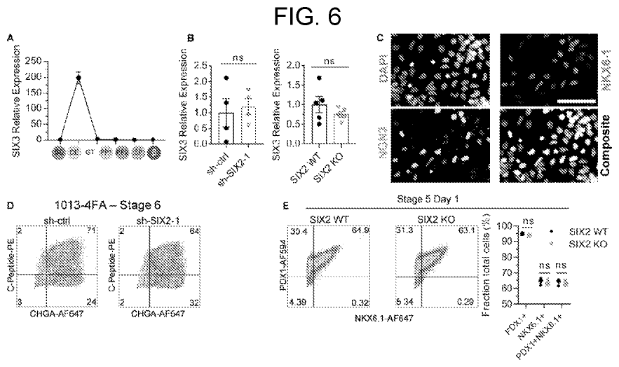

Since the KD studies supported a connection between SIX2 and the acquisition of SC-β cell function, CRISPR-Cas9 was used to KO SIX2 by deleting the SIX2 coding sequence to create 2 homozygous KO hESC lines (KO-SIX2-1 and KO-SIX-2) to ensure the complete absence of SIX2 (FIG. 5G-FIG. 5K). Similar to the KD studies, KO of SIX2 also resulted in significant reductions in both dynamic and static glucose-stimulated insulin secretion assays (FIG. 1F and FIG. 1G). In contrast to the KD studies, SIX2 KO resulted in no first- and second-phase dynamic insulin responses to high glucose and no response to glucose in the static assay. In addition, the overall amount of insulin secreted per DNA was 4.2±0.7 times lower in the dynamic assay and per cell was 6.2±1.5 times lower in the static assay at high glucose for SIX2 KO (FIG. 1F and FIG. 1G). Arda et al. (2016) showed that SIX3, a different transcription factor within the SIX family, is enriched in adult human islets relative to juvenile islets and that its expression is associated with increased β cell function. The expression profile of SIX3 was measured during the SC-β cell differentiation (FIG. 6A). While increased expression during early definitive endoderm induction was measured, SIX3 expression was low or undetected at the end of the protocol, and KD or KO of SIX2 did not alter SIX3 expression (FIG. 6B). These data establish SIX2 as a potent regulator of human β cell acquisition of functional maturation in vitro, demonstrating that SIX2 is necessary for first- and second-phase insulin secretion in response to glucose. SIX2 is not required for insulin production and secreting, but the lack of SIX2 reduced insulin secretion when cells are exposed to high glucose.

Characterization of SIX2 Expression During SC-β Cell Differentiation.

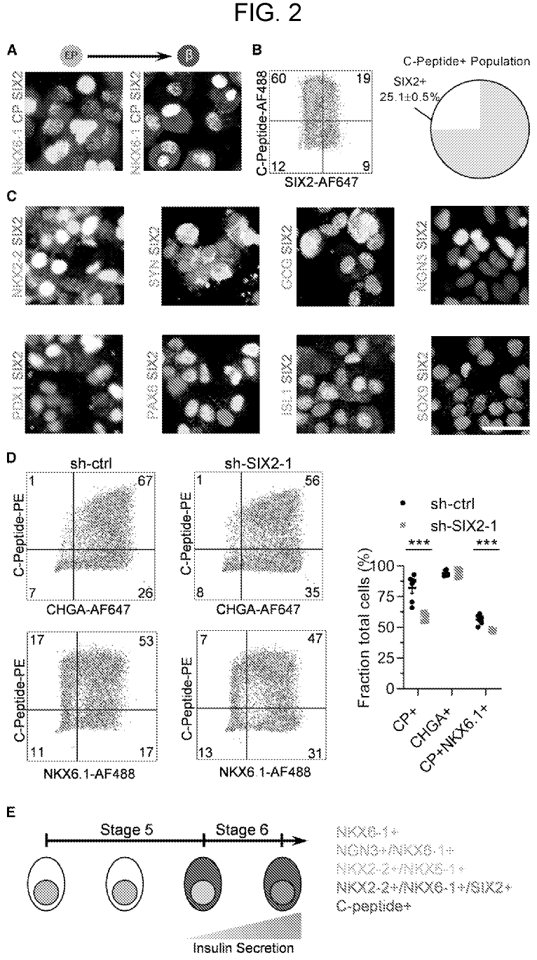

Next, the expression profile of SIX2 protein throughout the differentiation process was characterized. Using immunofluorescence, SIX2 expression in C-peptide⁺ cells were not detected at the beginning of stage 6, but after 11 days in stage 6, some C-peptide⁺ cells expressed nuclear SIX2 (FIG. 2A). Virtually all SIX2+ cells co-expressed NKX6-1 (FIG. 2A). With flow cytometry, it was demonstrated that 25.1%±0.5% of the C-peptide⁺ cells co-expressed SIX2 in stage 6 (FIG. 2B). However, some SIX2+ cells were observed outside the C-peptide⁺ population and are of unknown identity. It was also observed that SIX2 was restricted to NKX2-2⁺ and synaptophysin+(SYN) cells in stage 6 (FIG. 2C), indicating that the expression of SIX2 is restricted to this endocrine cell population. In spite of this, SIX2 was not observed in NGN3+ cells during stage 5 (FIG. 2C), even with many of these cells co-expressing NKX6-1 (FIG. 6C). Virtually all SIX2+ cells also co-expressed other pancreatic markers, such as ISL1, PAX6, and PDX1 (FIG. 2C). Furthermore, the α cell hormone-expressing glucagon⁺ (GCG) cells did not express SIX2, and SOX9⁺ progenitors were absent in the stage 6 population (FIG. 2C). KD of SIX2 reduced the fraction of cells expressing C-peptide⁺ and co-expressing C-peptide with NKX6.1 (FIG. 2D and FIG. 6D), demonstrating an effect on cell fate, while KO of SIX2 did not affect pancreatic progenitors (FIG. 6E). These data demonstrate that SIX2 protein expression is only detected in the endocrine population and influences final cell fate.

Transcriptional Profiling of SIX2 KD Cells.

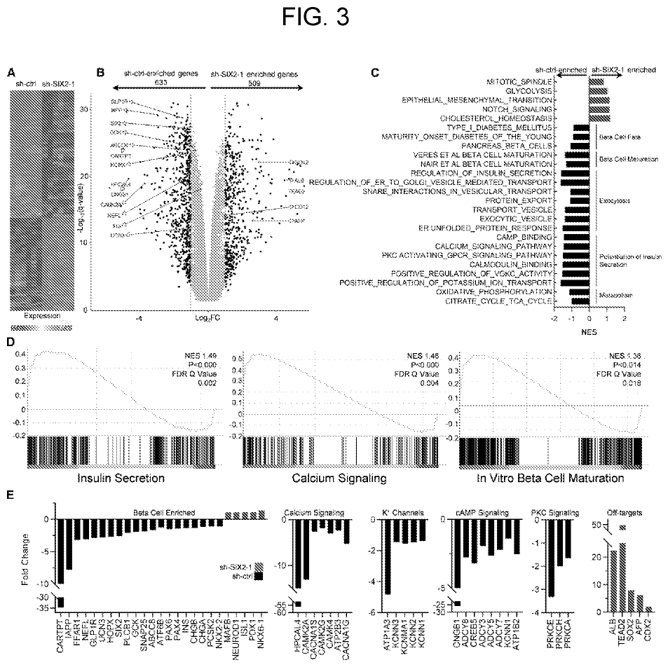

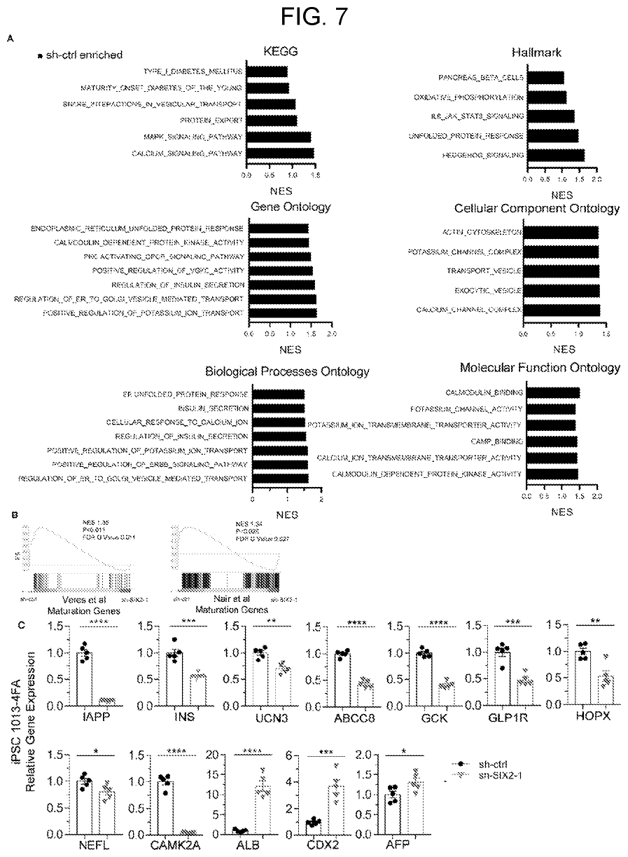

To further explore the role of SIX2 on SC-β cells, RNA sequencing was used to measure the transcriptome of stage 6 cells transduced with shRNA to KD SIX2 expression. A large number (10,421) of genes were significantly (adjusted p<0.05) affected by the KD of SIX2, including individual genes associated with β cell function and off-target non-3 cell genes (FIG. 3 and FIG. 7). Of the significantly different genes, 633 were enriched in the control cells and 509 were enriched in the SIX2 KD cells by at least a factor of 2 (FIG. 3B).

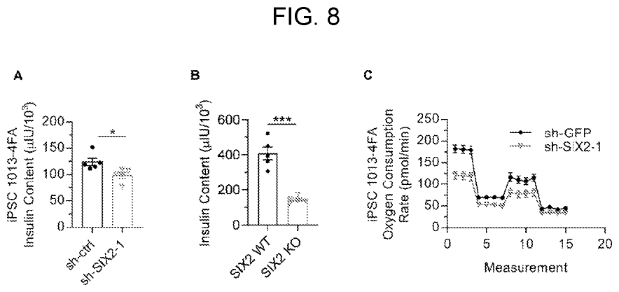

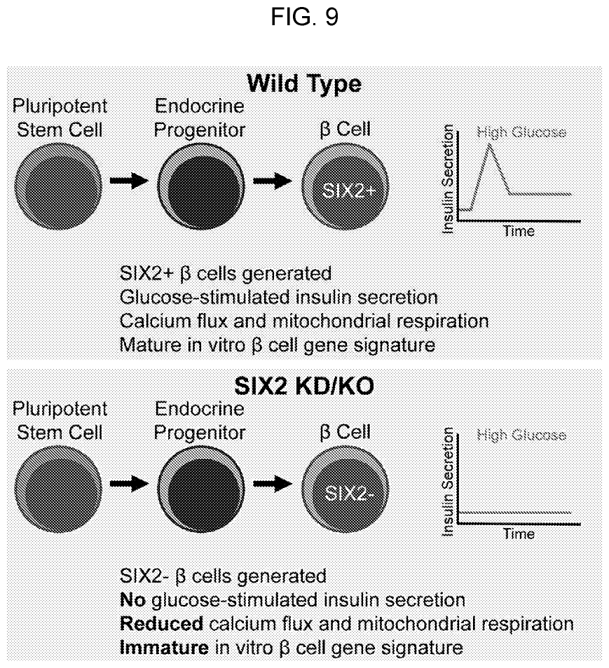

Several gene sets important for β cells, such as those correlated with β cell fate, β cell maturation, exocytosis, potentiation of insulin secretion, and maturation, were controlled by SIX2 (FIG. 3C-FIG. 3E, FIG. 7A, and FIG. 7B). The specific established gene sets that are well known included the gene sets β cell enriched (CARTPT, IAPP, GLP1R, GCK, ABCC8, PAX6, INS), calcium signaling (HPCAL4, CAMKK2A, CACNA1S), potassium channels (ATP1A3, KCNN3, KCNMA1), cyclic AMP (cAMP) signaling (ADCY8, CREB5, ADCY3), and protein kinase C (PKC) signaling (PRKC3, PRKCH, PRKCA). In addition, undesirable off-target markers, including liver (ALB, AFP), anterior endoderm (SOX2), and posterior endoderm (CDX2), and gene sets, such as glycolysis, were enriched in the SIX2 KD cells. RNA sequencing results were validated with a subset of relevant genes using real-time PCR in the 1013-4FA hiPSC background (FIG. 7C). The RNA sequencing data was compared to recent gene sets identified in Veres et al. (2019) and Nair et al. (2019) as positively correlating with time in the final stage of differentiation of SC-β cells in vitro. It was found that the SIX2 KD data were statistically associated with the Veres et al. and Nair et al. gene sets (FIG. 3C, FIG. 3D, FIG. 7B, and FIG. 8). Specifically, the KD of SIX2 repressed many of the genes that increased expression in Veres et al. (2019) and Nair et al. (2019), suggesting that many of these identified genes are controlled by SIX2. These data support the conclusion that SIX2 controls the expression of many β cell genes related to normal physiological function and differentiation.

Physiological Profiling of SIX2 KD SC-β Cells.

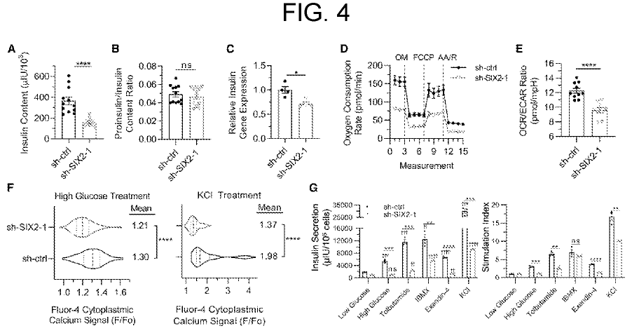

Key β cell processes implicated by the SIX2 KD RNA sequencing data were studied. First, it was found that KD of SIX2 reduced insulin content and INS gene expression, but it did not affect insulin processing (FIG. 4A-FIG. 4C and FIG. 8A). Second, mitochondrial respiration and glycolysis were assessed because switching from glycolysis to mitochondrial respiration is necessary for the maturation and normal physiological function of β cells (Nair et al., 2019). The Seahorse XFe24 extracellular flux assay was used to measure the oxygen consumption rate (OCR) and the extracellular acidification rate (ECAR), respectively. KD of SIX2 resulted in a decreased OCR and ratio of OCR to ECAR (FIG. 4D, FIG. 4E, and FIG. 8C). Cytoplasmic calcium flux was also evaluated, as the influx of calcium is necessary before insulin secretion and is triggered by glucose-induced depolarization in β cells (Rutter and Hodson, 2013). KD of SIX2 slightly decreased glucose-stimulated and greatly decreased KCl-stimulated increases in cytoplasmic calcium as determined with Fluo-4 AM stained cells (FIG. 4F). These data are consistent with decreased glucose-stimulated insulin secretion (FIG. 1D-FIG. 1G and FIG. 5C-FIG. 5F) and the RNA sequencing analysis (FIG. 3), indicating that SIX2 plays a key role in the metabolism and upstream signaling relating to the functional maturation of SC-β cells.

Finally, other known insulin secretagogues were explored, observing that while SIX2 KD cells were able to respond to all treatments, the amount of insulin secretion was much lower than the control (FIG. 4G). Stimulation with KCl and 3-isobutyl-1-methylxanthine (IBMX) demonstrated that SIX2 KD cells were capable of elevating insulin secretion, but glucose-dependent secretion was severely impaired without SIX2. Furthermore, considering the large amount of insulin secretion with treatments targeting downstream processes, particularly depolarization (KCl) and cAMP accumulation (IBMX), upstream mechanisms appear more affected by SIX2 KD, namely glucose sensing and metabolism. In addition to defects in glucose-stimulated insulin secretion, SC-β cells with KD of SIX2 have defects in insulin content, mitochondrial respiration, calcium signaling, and response to a wide array of secretagogues.

### Discussion

Here, it was demonstrated that SIX2 influences the generation human SC-β cells in vitro. Increases in SIX2 expression correlates with increases in insulin secretion as SC-β cells mature during stage 6 of the differentiation protocols. KD or KO of SIX2 dramatically reduces glucose-stimulated insulin secretion, including first- and second-phase dynamic insulin release and the total amount of insulin released from the cells. Expression of SIX2 protein appears to be restricted to endocrine cells. RNA sequencing of cells with the KD of SIX2 reveals that a large number of gene sets associated with β cells and off-targets are negatively affected, including recently defined gene sets of maturing SC-β cells. The physiological effects of many of these gene sets were confirmed by measuring reductions in insulin content, insulin gene expression, mitochondrial respiration, calcium flux, and insulin secretion in response to compounds that block the K^{ATP} channel (tolbutamide), accumulate cAMP (IBMX), activate GLP1R (Exendin-4), and depolarize the membrane (KCl).

A major goal in regenerative medicine is to generate fully mature replacement cells differentiated from stem cells. However, while many differences often exist in the function and gene expression of stem cell-differentiated cells, often referred to as the maturation phenotype specific to the differentiated cells in question, identifying specific parameters on which to focus is often difficult due to a lack of understanding of human developmental biology. In the case of SC-β cells, many genes and pathways have been focused on and studied in the context of improving these cells, including YAP (Rosado-Olivieri et al., 2019), the ROCKII pathway (Ghazizadeh et al., 2017), the transforming growth factor β (TGF-β) pathway (Velazco-Cruz et al., 2019), and the cytoskeleton (Hogrebe et al., 2020). Further studies have connected diabetic pathogenetic variants with impairments in polyhormonal endocrine or SC-β cells, including INS (Balboa et al., 2018, Ma et al., 2018), HNF1-α (Cardenas-Diaz et al., 2019), WFS1 (Maxwell et al., 2020, Shang et al., 2014), ZNT8 (Dwivedi et al., 2019), NEUROD1 (Romer et al., 2019), and GCK (Hua et al., 2013).

In contrast, focused on here is the role of SIX2 in the differentiation to and maturation of SC-β cells and to establish a critical connection between this transcription factor and the generation and functional maturation of SC-β cells, particularly regarding glucose-stimulated insulin secretion. Arda et al. (2016) studied both SIX2 and SIX3 in the human insulinoma EndoC-PH1 cell line and found overexpressing SIX3 but not SIX2 to increase insulin secretion and content in addition to increased expression of these genes in adult versus juvenile islets, supporting differing roles for these transcription factors. This study differs from that of Arda et al. (2016) in several respects. SIX2 in the context of differentiating and maturing SC-β cells was studied, the process of which has increasing SIX2 expression as cells mature with time, a considerably different developmental context than that modeled by EndoC-PH1 cells. Furthermore, this study investigates many other aspects of β cell phenotype not explored by Arda et al., including demonstrating that both first- and second-phase dynamic insulin secretion are eliminated with KO of SIX2, made possible by recent discoveries of generating SC-β cells using these functional characterizations (Velazco-Cruz et al., 2019), indexing transcriptional changes with KD of SIX2, and showing how mechanisms of β cell glucose sensing, respiration, and calcium flux are disrupted with KD of SIX2. Furthermore, while this data demonstrate the importance of SIX2 in SC-β cells, this transcription factor is in the presence of many other transcription factors that are important for the β cell phenotype, including PDX1, NKX6-1, NKX2-2, and NEUROD1 (Hogrebe et al., 2020). Understanding the molecular interactions and regulatory network of SIX2 with these transcription factors would be valuable in future studies.

Differences in transcriptional regulation in rodent and human β cells are well known (Benner et al., 2014). Because SIX2 expression is restricted to human β cells (Segerstolpe et al., 2016, Xin et al., 2016), focused studies on human cell model systems, such as that provided by this in vitro differentiation platform, are essential in the investigation of cell maturation and disease. Proper understanding of the molecular mechanisms that control human β cell maturation is necessary for developing further improvements in SC-β cell technologies for diabetes cell-replacement therapies. As only a fraction of C-peptide⁺ cells currently express SIX2, increased co-expression could result in differentiated populations with increased function and utility for cell therapy. Further study into the role of SIX2 could reveal new insights into increasing the functional maturation of SC-β cells and the regulation of expression of other β cell genes, such as MAFA (Nair et al., 2019, Velazco-Cruz et al., 2019), or β cell failure in type 2 diabetes.

Methods

Base Media.

| Reagent | S1 | | S2 | | S3 | | S5 | | S6 | | BE1 | | BE2 | | BE3 | |
|---|---|---|---|---|---|---|---|---|---|---|---|---|---|---|---|---|
| MCDB131 | 500 | mL | 500 | mL | 500 | mL | 500 | mL | 500 | mL | 500 | mL | 500 | mL | 500 | mL |
| Glucose | 0.22 | g | 0.22 | g | 0.22 | g | 1.8 | g | 0.23 | g | 0.8 | g | 0.4 | g | 0.22 | g |
| NaHCO₃ | 1.23 | g | 0.615 | g | 0.615 | g | 0.877 | g | N/A | | 0.587 | g | 0.587 | g | 0.877 | g |
| FAF-BSA | 10 | g | 10 | g | 10 | g | 10 | g | 10.5 | g | 0.5 | g | 0.5 | g | 10 | g |
| ITS-X | 10 | μL | 10 | μL | 2.5 | mL | 2.5 | mL | N/A | | N/A | | N/A | | 2.5 | mL |
| Glutamax | 5 | mL | 5 | mL | 5 | mL | 5 | mL | 5.2 | mL | 5 | mL | 5 | mL | 5 | mL |

| | | | | | | | | | | | | | | |
|---|---|---|---|---|---|---|---|---|---|---|---|---|---|---|
| Vitamin C | 22 | mg | 22 | mg | 22 | mg | 22 | mg | N/A | N/A | 22 | mg | 22 | mg |

| Heparin | N/A | | N/A | | N/A | | 5 | mg | 10 | μg/mL | N/A | N/A | N/A |
|---|---|---|---|---|---|---|---|---|---|---|---|---|---|
| Pen/Strep | 5 | mL | 5 | mL | 5 | mL | 5 | mL | 5.2 | mL | N/A | N/A | N/A |

| | | | | | | | | | |
|---|---|---|---|---|---|---|---|---|---|
| Non-Essential | N/A | N/A | N/A | N/A | 5.2 | mL | N/A | N/A | N/A |
| Amino Acids | | | | | | | | | |
| ZnSO₄ | N/A | N/A | N/A | N/A | 1 | μM | N/A | N/A | N/A |
| Trace | N/A | N/A | N/A | N/A | 523 | μL | N/A | N/A | N/A |
| Elements A | | | | | | | | | |
| Trace | N/A | N/A | N/A | N/A | 523 | μL | N/A | N/A | N/A |
| Elements B | | | | | | | | | |

Factors.

| Compound | Company | Part # |
|---|---|---|
| Activin A | R&D Systems | 338-AC |
| CHIR99021 | Stemgent | 04-0004-10 |
| KGF | Peprotech | AF-100-19 |
| LDN193189 | Reprocell | 40074 |
| PdBU | MilliporeSigma | 524390 |
| Retinoic Acid | MilliporeSigma | R2625 |
| SANT1 | MilliporeSigma | S4572 |
| Y27632 | Abcam | ab120129 |
| Latrunculin A | Cayman Chemical | 10010630 |
| Alk5 inhibitor type II | Enzo Life Sciences | ALX-270-445-M005 |
| Betacellulin | R&D Systems | 261-CE-050 |
| T3 | Biosciences | 64245 |
| XXI | MilliporeSigma | 595790 |
| TPPB | Tocris | 53431 |

Protocol 1.

| | Day | Media | Factor | Final Concentration | |
|---|---|---|---|---|---|
| Stage 0 | 1 | mTeSR1 | Y27632 | 10 | μM |
| | 2 | No Change | | | |
| | 3 | mTeSR1 | | | |
| Stage 1 | 4 | S1 | Activin A | 100 | ng/mL |
| | | | CHIR99021 | 3 | μM |
| | 5 | ! | Activin A | 100 | ng/mL |
| | 6 | No Change | | | |
| Stage 2 | 7 | S2 | KGF | 50 | ng/mL |
| | 8 | S2 | KGF | 50 | ng/mL |
| | 9 | No Change | | | |
| Stage 3 | 10 | S3 | LDN193189 | 200 | nM |
| | | | KGF | 50 | ng/mL |
| | | | SANT1 | 0.25 | μM |
| | | | RA | 2 | μM |
| | | | PdbU | 0.5 | μM |
| | | | Y27632 | 10 | μM |
| Stage 4 | 11 | S4 | KGF | 50 | ng/mL |
| | | | SANT1 | 0.25 | μM |
| | | | RA | 0.1 | μM |
| | | | Y27632 | 10 | μM |
| | | | Activin A | 5 | ng/mL |
| | 12 | S4 | KGF | 50 | ng/mL |
| | | | SANT1 | 0.25 | μM |
| | | | RA | 0.1 | μM |
| | | | Y27632 | 10 | μM |
| | | | Activin A | 5 | ng/mL |
| | 13 | No Change | | | |
| | 14 | S4 | KGF | 50 | ng/mL |
| | | | SANT1 | 0.25 | μM |
| | | | RA | 0.1 | μM |
| | | | Y27632 | 10 | μM |
| | | | Activin A | 5 | ng/mL |
| | 15 | No Change | | | |
| Stage 5 | 16 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Betacellulin | 20 | ng/mL |
| | 17 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Betacellulin | 20 | ng/mL |
| | 18 | No Change | | | |
| | 19 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Betacellulin | 20 | ng/mL |
| | 20 | No Change | | | |
| | 21 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Betacellulin | 20 | ng/mL |
| | 22 | No Change | | | |

| | |
|---|---|
| Stage 6 | Feed S6 every other day |

Protocol 2.

| | Day | Media | Factor | Final Concentration | |
|---|---|---|---|---|---|
| Stage 0 | 1 | mTeSR1 | Y27632 | 10 | μM |
| Stage 1 | 2 | BE1 | Activin A | 100 | ng/mL |
| | | | CHIR99021 | 3 | μM |
| Stage 1 | 3 | BE1 | Activin A | 100 | ng/mL |
| Stage 1 | 4 | BE1 | Activin A | 100 | ng/mL |
| Stage 1 | 5 | BE1 | Activin A | 100 | ng/mL |
| Stage 2 | 6 | BE2 | KGF | 50 | ng/mL |
| | 7 | BE2 | KGF | 50 | ng/mL |
| Stage 3 | 8 | BE3 | LDN193189 | 200 | nM |
| | | | KGF | 50 | ng/mL |
| | | | SANT1 | 0.25 | μM |
| | | | RA | 2 | μM |
| | | | TPPB | 500 | nM |
| Stage 3 | 9 | BE3 | LDN193189 | 200 | nM |
| | | | KGF | 50 | ng/mL |
| | | | SANT1 | 0.25 | μM |
| | | | RA | 2 | μM |
| | | | TPPB | 500 | nM |
| Stage 4 | 10 | BE3 | KGF | 50 | ng/mL |
| | | | SANT1 | 0.25 | μM |
| | | | RA | 0.1 | μM |
| | | | TPPB | 500 | nM |
| | | | LDN193189 | 200 | nM |
| | 11 | BE3 | KGF | 50 | ng/mL |
| | | | SANT1 | 0.25 | μM |
| | | | RA | 0.1 | μM |
| | | | TPPB | 500 | nM |
| | | | LDN193189 | 200 | nM |
| | 12 | BE3 | KGF | 50 | ng/mL |
| | | | SANT1 | 0.25 | μM |
| | | | RA | 0.1 | μM |
| | | | TPPB | 500 | nM |
| | | | LDN193189 | 200 | nM |
| | 13 | BE3 | KGF | 50 | ng/mL |
| | | | SANT1 | 0.25 | μM |
| | | | RA | 0.1 | μM |
| | | | TPPB | 500 | nM |
| | | | LDN193189 | 200 | nM |
| Stage 5 | 14 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Latrunculin | | |
| | | | A | 1 | μM |
| | | | Betacellulin | 20 | ng/mL |
| | 15 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Betacellulin | 20 | ng/mL |
| | 16 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Betacellulin | 20 | ng/mL |
| | 17 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Betacellulin | 20 | ng/mL |
| | 18 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Betacellulin | 20 | ng/mL |
| | 19 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Betacellulin | 20 | ng/mL |
| | 20 | S5 | SANT1 | 0.25 | μM |
| | | | RA | 0.1 | μM |
| | | | XXI | 1 | μM |
| | | | Alk5i | 10 | μM |
| | | | T3 | 1 | μM |
| | | | Betacellulin | 20 | ng/mL |

| | |
|---|---|
| Stage 6 | Feed S6 every other day |

Antibodies.

| Antibody Target | Company | Part # | Dilution |
|---|---|---|---|
| C-peptide | DSHB | GN-ID4-S | 1:300 |
| NKX6-1 | DSHB | F55A12-S | 1:100 |
| Glucagon | Abcam | ab82270 | 1:300 |
| PDX1 | R&D Systems | AF2419 | 1:300 |
| PAX6 | BDBiosciences | 561462 | 1:300 |
| CHGA | Abcam | ab15160 | 1:1000 |
| ISL1 | DSHB | 40.2d6-s | 1:300 |
| SIX2 | Proteintech | 11562-1-AP | 1:2000 |
| NGN3 | R&D Systems | AF3444 | 1:300 |
| NKX2-2 | DSHB | 74.5A5 | 1:300 |
| Synaptophysin | LifeSpan | LS-C174787 | 1:100 |
| | BioSciences | | |
| SOX9 | Invitrogen | 14-9765-80 | 1:300 |
| anti-rat-alexa fluor 488 | Invitrogen | a21208 | 1:300 |
| anti-mouse-alexa fluor 647 | Invitrogen | a31571 | 1:300 |
| anti-rabbit-alexa fluor 647 | Invitrogen | a31573 | 1:300 |
| anti-goat-alexa fluor 647 | Invitrogen | a21447 | 1:300 |
| antirabbit-alexa fluor 488 | Invitrogen | a21206 | 1:300 |
| anti-mouse-alexa fluor 594 | Invitrogen | a21203 | 1:300 |
| anti-rabbit-alexa fluor 594 | Invitrogen | a21207 | 1:300 |
| anti-goat-alexa fluor 594 | Invitrogen | a11058 | 1:300 |
| anti-rat-PE | Jackson | 712-116-153 | 1:300 |
| | Immuneresearch | | |
| anit-sheep-alexa fluor 594 | Invitrogen | a11016 | 1:300 |

DSHB = Developmental Studies Hybridoma Bank

Oligonucleotides.

| | SEQ ID NO: | SEQ ID NO: | | |
|---|---|---|---|---|
| REAGENT or RESOURCE | (Forward) | (Reverse) | SOURCE | IDENTIFIER |
| qPRC-INS-F, R: | 1 | 2 | Velazco-Cruz | N/A |
| CAATGCCACGCTTCTGC, | | | et al., 2019 | |
| TTCTACACACCCAAGACCCG | | | | |
| qPRC-TBP-F, R: | 3 | 4 | Velazco-Cruz | N/A |
| GCCATAAGGCATCATTGGAC, | | | et al., 2019 | |
| AACAACAGCCTGCCACCTTA | | | | |
| qPRC-SIX2-F, R: | 5 | 6 | This study | N/A |
| AAGGCACACTACATCGAGGC, | | | | |
| CACGCTGCGACTCTTTTCC | | | | |
| qPRC-SIX3-F, R: | 7 | 8 | This study | N/A |
| CTGCCCACCCTCAACTTCTC, | | | | |
| GCAGGATCGACTCGTGTTTGT | | | | |
| qPRC-IAPP-F, R: | 9 | 10 | This study | N/A |
| ACATGTGGCAGTGTTGCATT, | | | | |
| TCATTGTGCTCTCTGTTGCAT | | | | |
| qPRC-UCN3-F, R: | 11 | 12 | Velazco-Cruz | N/A |
| TGTAGAACTTGTGGGGGAGG, | | | et al., 2019 | |
| GGAGGGAAGTCCACTCTCG | | | | |
| qPRC-ABCC8-F, R: | 13 | 14 | This study | N/A |
| GCCCACGAAAGTTATGAGGA, | | | | |
| AAGGAGATGACCAGCCTCAG | | | | |
| qPRC-GCK-F, R: | 15 | 16 | Velazco-Cruz | N/A |
| ATGCTGGACGACAGAGCC, | | | et al., 2019 | |
| CCTTCTTCAGGTCCTCCTCC | | | | |
| qPRC-GLP1R1-F, R: | 17 | 18 | This study | N/A |
| GGTGCAGAAATGGCGAGAATA, | | | | |
| CCGGTTGCAGAACAAGTCTGT | | | | |
| qPRC-HOPX-F, R: | 19 | 20 | This study | N/A |
| GAGACCCAGGGTAGTGATTTGA, | | | | |
| AAAAGTAATCGAAAGCCAAGCAC | | | | |
| qPRC-NEFL-F, R: | 21 | 22 | This study | N/A |
| ATGAGTTCCTTCAGCTACGAGC, | | | | |
| CTGGGCATCAACGATCCAGA | | | | |
| qPRC-CAMK2A-F, R: | 23 | 24 | This study | N/A |
| GCTCTTCGAGGAATTGGGCAA, | | | | |
| CCTCTGAGATGCTGTCATGTAGT | | | | |
| qPRC-ALB-F, R: | 25 | 26 | This study | N/A |
| CCTTTGGCACAATGAAGTGGGTAACC, | | | | |
| CAGCAGTCAGCCATTTCACCATAGG | | | | |
| qPRC-CDX2-F, R: | 27 | 28 | This study | N/A |
| CCTCTGAGATGCTGTCATGTAGT, | | | | |
| GGTGATGTAGCGACTGTAGTGAA | | | | |
| qPRC-AFP-F, R: | 29 | 30 | Hogrbe et | N/A |
| TGTACTGCAGAGATAAGTTTAGCTGAC, | | | al., 2020 | |
| CCTTGTAAGTGGCTTCTTGAACA | | | | |
| PCR-Deletion Primers-F, R: | 31 | 32 | This study | N/A |
| GGGAGAACGAGTGAGAAGCG, | | | | |
| TGCGGGTCTTTCAGTACCTG | | | | |
| PCR-Inside Primers-F, R: | 33 | 34 | This study | N/A |
| CAGTTCTGGGAGAGAAGAGAC, | | | | |
| GGGCTGGATTCTGTTCCCATA | | | | |

*F = Forward Primer
*R = Reverse Primer

| | | SEQ. | | |
|---|---|---|---|---|
| | | ID | | |
| | | NO.: | | |
| sh-ctrl | GCGCGATCACATGGTCCTGCT | 35 | This study | N/A |
| (GFP): | | | | |
| sh-SIX2-1 | CAACGAGAACTCCAATTCTAA | 36 | This study | N/A |
| (human): | | | | |
| sh-SIX2-2 | GAGCACCTTCACAAGAATGAA | 37 | This study | N/A |
| (human): | | | | |

| | | |
|---|---|---|
| REAGENT or | | |
| RESOURCE | SOURCE | IDENTIFIER |

Antibodies

| | | |
|---|---|---|
| Rat anti C-peptide | DSHB | Cat#GN-ID4; RRID:AB_2255626 |
| Mouse anti NKX6-1 | DSHB | Cat#F55A12; RRID:AB_532379 |
| Mouse anti | ABCAM | Cat#ab82270; RRID:AB_1658481 |
| Glucagon | | |
| Goat anti PDX1 | R&D Systems | Cat#AF2419; RRID:AB_355257 |
| Mouse anti PAX6 | BDBiosciences | Cat#561462; RRID:AB_10715442 |
| Rabbit anti CHGA | ABCAM | Cat#ab15160 |
| Mouse anti ISL1 | DSHB | Cat#40.2D6; RRID:AB_528315 |
| Rabbit anti SIX2 | Proteintech | Cat#11562-1-AP; RRID:AB_2189084 |
| Sheep anti NGN3 | R&D Systems | Cat#AF3444; RRID:AB_2149527 |
| Mouse anti NKX2-2 | DSHB | Cat#74.5A5; RRID:AB_531794 |
| Mouse anti | LifeSpan | Cat#LS-C174787; RRID:AB_2811021 |
| Synaptophysin | BioSciences | |
| Mouse anti SOX9 | Invitrogen | Cat#14-9765-80; RRID:AB_2573005 |
| anti-rat-alexa fluor | Invitrogen | Cat#A-21208; RRID:AB_141709 |
| 488 | | |
| anti-mouse-alexa | Invitrogen | Cat#a31571; RRID:AB_162542 |
| fluor 647 | | |
| anti-rabbit-alexa | Invitrogen | Cat#a31573; RRID:AB_2536183 |
| fluor 647 | | |
| anti-goat-alexa | Invitrogen | Cat#a21447; RRID:AB_141844 |
| fluor 647 | | |
| anti-rabbit-alexa | Invitrogen | Cat#a21206; RRID:AB_2535792 |
| fluor 488 | | |
| anti-mouse-alexa | Invitrogen | Cat#a21203; RRID:AB_141633 |
| fluor 594 | | |
| anti-rabbit-alexa | Invitrogen | Cat#a21207; RRID:AB_141637 |
| fluor 594 | | |
| anti-goat-alexa | Invitrogen | Cat#a11058; RRID:AB_2534105 |
| fluor 594 | | |
| anti-rat-PE | Jackson | Cat#712-116-153; RRID:AB_2340657 |
| | Immuneresearch | |
| anti-sheep-alexa | Invitrogen | Cat#a11016; RRID:AB_10562537 |
| fluor 594 | | |

Chemicals, Peptides, and Recombinant Proteins

| | | |
|---|---|---|
| Lenti-X | Takara | Cat#631232 |
| concentrator | | |
| mTeSR1 | StemCell | Cat#05850 |
| | Technologies | |
| Accutase | StemCell | Cat#07920 |
| | Technologies | |
| Y27632 | Abcam | Cat#ab120129 |
| Matrigel | Corning | Cat#356230 |
| TrypLE | Life Technologies | Cat#12-604-039 |
| Dnase | QIAGEN | Cat#79254 |
| HEPES | GIBCO | Cat#15630-080 |
| Extendin-4 | MilliporeSigma | Cat#E7144 |
| IBMX | MilliporeSigma | Cat#I5879 |
| Tolbutamide | MilliporeSigma | Cat#T0891 |
| KCI | ThermoFisher | Cat#BP366500 |
| Tris | MilliporeSigma | Cat#T6066 |
| EDTA | Ambion | Cat#327371000 |
| Paraformaldehyde | Electron | Cat#15714 |
| | Microscopy | |
| | Science | |
| Donkey Serum | Jackson | Cat#017-000-121 |
| | Immunoresearch | |
| Triton X-100 | Acros Organics | Cat#327371000 |
| RPMI-1640 | Sigma | Cat#R6504 |
| Oligomycin | Calbiochem | Cat#1404-19-9 |
| FCCP | Sigma | Cat#270-86-5 |
| Rotenone | Calbiochem | Cat#83-79-4 |
| Antimycin A | Sigma | Cat#1397-94-0 |
| Fluo-4 AM | Invitrogen | Cat#F14201 |
| DMEM | MilliporeSigma | Cat#D6429 |
| HI Fetal Bovine | MilliporeSigma | Cat#F4135 |
| Serum | | |
| Opti-MEM | Life Technologies | Cat#31985-070 |
| Polyethylenimine | Polysciences | Cat#24765-2 |
| ‘Max’ MW 40,000 | | |
| Da | | |

Commercial Assays

| | | |
|---|---|---|
| Human Insulin Elisa | ALPCO | Cat#80-INSHU-E10.1; RRID:AB_2801438 |
| Proinsulin ELISA | Mercodia | Cat#10-1118-01; RRID:AB_2754550 |
| Quant-iT Picogreen | MilliporeSigma | Cat#T6066 |
| dsDNA assay kit | | |
| Lenti-X qRT-PCR | Takara | Cat#631235 |
| Titration Kit | | |
| RNeasy Mini Kit | QIAGEN | Cat#74016 |
| High Capacity cDNA | Applied | Cat#4368814 |
| Reverse | Biosystems | |
| Transcriptase Kit | | |
| PowerUp SYBR | Applied | Cat#A25741 |
| Green Master Mix | Biosystems | |

Deposited Data

| | | |
|---|---|---|
| Raw and Analyzed | Here | GEO: GSE147737 |
| RNA Seq Data | | |

Experimental Models: Cell Lines

| | | |
|---|---|---|
| Human: HUES8 | HSCI | hES Cell line: HUES-8 |
| hESC | | |
| Human: iPSC 1013 | HSCI | hiPS Cell line: 1013-4FA |
| Lenti-X 293T | Takara | Cat#632180 |

Recombinant DNA

| | | |
|---|---|---|
| sh-ctrl | RNAi Core, | pLKO.1 shGFP, GCGCGATCACATGGTCCTGCT (SEQ ID |
| | Washington | NO: 35) |
| | University in St. | |
| | Louis | |
| sh-SIX2-l | RNAi Core, | pLKO.1 TRC sh-SIX2, CAACGAGAACTCCAATTCTAA (SEQ |
| | Washington | ID NO: 36) |
| | University in St. | |
| | Louis | |
| sh-SIX2-2 | RNAi Core, | pLKO.1 TRC sh-SIX2, GAGCACCTTCACAAGAATGAA (SEQ |
| | Washington | ID NO: 37) |
| | University in St. | |
| | Louis | |
| psPAX2 | Gift from Didier | Addgene Plasmid Cat#12260; RRID:Addgene_12260; |
| | Trono | http://n2t.net/addgene:12260 |
| pMD2.G | Gift from Didier | Addgene Plasmid Cat#12259; RRID:Addgene_12259; |
| | Trono | http://n2t.net/addgene:12259 |

Software and Algorithms

| | | |
|---|---|---|
| Fiji ImageJ | ImageJ public | https://imagej.net/Fiji/Downloads |
| | freeware | |
| PRISM8 | GraphPad | https://www.graphpad.com/scientific-software/prism/ |
| FLOWJO | FLOWJO | https://www.flowjo.com/solutions/flowjo/downloads |
| GSEA | GSEA | https://www.gsea-msigdb.org/gsea/index.jsp |

Other

| | | |
|---|---|---|
| 30-mL spinner | Reprocell | Cat#ABBWVS03A |
| flasks | | |
| Vi-Cell XR | Beckman Coulter | Cat#Vi-Cell XR |
| Tanswells | Corning | Cat#431752 |
| Seahorse Xfe24 Flux | Agilent | Cat#Xfe24 |
| analyzer | | |
| #1.5 glass bottom | Cellvis | Cat#963-1.5H-N |
| 96 well plate | | |
| 8-channel | ISMATEC | Cat#ISM931C |
| peristaltic pump | | |
| Inlet/outlet two- | ISMATEC | Cat#070602-04i-ND |
| stop tubing | | |
| Cell chamber | BioRep | Cat#Peri-Chamber |
| Dispensing nozzle | BioRep | Cat#Peri-Nozzle |
| Connection tubbing | BioRep | Cat#Peri-TUB-040 |
| Bio-Gel P-4 | Bio-Rad | Cat#150-4124 |

Materials Availability

shRNA plasmids used in this study are from the TRC shRNA library and available from the RNAi Core at Washington University in St. Louis. HUES8 cell line is available through the Harvard Stem Cell Institute (HCSI). SIX2 KO cell lines are made available upon request to Lead Contact.

Data and Code Availability

The RNA sequencing data generated in this study is made available at the Gene Expression Omnibus (GEO). The accession number for the raw and processed data reported here is GEO: GSE147737.

Experimental Model and Subject Details

Culture of Undifferentiated hESCs and Differentiation to Stage 6 Cells.

Cell culture was performed as previously described (Hogrebe et al., 2020, Millman et al., 2016, Velazco-Cruz et al., 2019). This work was performed with the approval of the Washington University School of Medicine Embryonic Stem Cell Research Oversight Committee (ESCRO). The HUES8 hESC and 1013-4FA hiPSC lines were generously provided by Dr. Douglas Melton (Harvard University) and have been published previously (Hogrebe et al., 2020, Velazco-Cruz et al., 2019). All data is with the HUES8 cell line unless otherwise noted to be 1013-4FA. For differentiation protocol 1 (Velazco-Cruz et al., 2019), which was used unless otherwise noted, undifferentiated HUES8 were cultured in mTeSR1 (StemCell Technologies; 05850) in 30-mL spinner flasks (REPROCELL; ABBWVS03A) on a rotator stir plate (Chemglass) at 60 RPM in a humidified 37° C. 5% CO₂ tissue culture incubator. Stem cells were passaged every 3 days by single cell dispersion using Accutase (StemCell Technologies; 07920), viable cells counted with Vi-Cell XR (Beckman Coulter), and seeded at 6×10⁵ cells/mL in mTeSR1+ 10 μM Y27632 (Abcam; ab120129). The media was then changed as outlined in the methods section to induce differentiation. For differentiation protocol 2 (Hogrebe et al., 2020), which was used for the KO studies and 1013-4FA differentiations, undifferentiated pluripotent stem cells were cultured in mTeSR1 on plates coated with Matrigel (Corning; 356230) in a humidified 37° C. 5% CO₂ tissue culture incubator. Stem cells were passaged every 4 days single cell dispersion using TrypLE (Life Technologies; 12-604-039), viable cells counted with Vi-Cell XR, and seeded at 5.2×10⁵ cells/cm² in mTeSR1+ 10 μM Y27632. The media was then changed as outlined in the methods section to induce differentiation. On stage 6 day 1 of protocol 1 and stage 6 day 7 of protocol 2 cells were single cell dispersed using TrypLE (Life Technologies; 12-604-039; 15-minute incubation for protocol 1, 6-minute incubation for protocol 2). Following single cell dispersion cells were re-aggregated by seeding 5 million cells in 5 mL of stage 6 media into a well of a 6-well plate placed on an orbi-shaker (Benchmark) rotating at 100 RPM; clusters were allowed to re-aggregate and media was changed 48 hours post seeding.

Method Details

Real-Time PCR.

Measurements were performed as previously described (Velazco-Cruz et al., 2019). RNeasy Mini Kit (QIAGEN; 74016) with DNase treatment (QIAGEN; 79254) was used for RNA extraction. High Capacity cDNA Reverse Transcriptase Kit (Applied Biosystems; 4368814) was used to make cDNA. PowerUp SYBR Green Master Mix (Applied Biosystems; A25741) on a StepOnePlus (Applied Biosystems) was used to perform the real-time PCR reactions. Melting curves were ran for each primer to determine specificity of amplification. ΔΔCt methodology with TBP normalization was used for analysis. For samples undetected using real-time PCR CT values were set to 40 in the analysis. Primer sequences used were (gene, forward primer, reverse primer):

| | Forward | | Reverse | |
|---|---|---|---|---|
| | Primer | | Primer | Reverse |
| | SEQ | | SEQ | Primer |
| Gene | ID NO: | Forward Primer | ID NO: | Sequence |
| INS | 1 | CAATGCCACGCTTC | 2 | TTCTACACAC |
| | | TGC | | CCAAGACCCG |
| TBP | 3 | GCCATAAGGCATCA | 4 | AACAACAGCC |
| | | TTGGAC | | TGCCACCTTA |
| SIX2 | 5 | AAGGCACACTACAT | 6 | CACGCTGCGA |
| | | CGAGGC | | CTCTTTTCC |
| SIX3 | 7 | CTGCCCACCCTCAA | 8 | GCAGGATCGA |
| | | CTTCTC | | CTCGTGTTTG |
| | | | | t |
| IAPP | 9 | ACATGTGGCAGTGT | 10 | TCATTGTGCT |
| | | TGCATT | | CTCTGTTGCA |
| | | | | T |
| UCN3 | 11 | TGTAGAACTTGTGG | 12 | GGAGGGAAGT |
| | | GGGAGG | | CCACTCTCG |
| ABCC8 | 13 | GCCCACGAAAGTTA | 14 | AAGGAGATGA |
| | | TGAGGA | | CCAGCCTCAG |
| GCK | 15 | ATGCTGGACGACAG | 16 | CCTTCTTCAG |
| | | AGCC | | GTCCTCCTCC |
| GLP1R1 | 17 | GGTGCAGAAATGGC | 18 | CCGGTTGCAG |
| | | GAGAATA | | AACAAGTCTG |
| | | | | T |
| HOPX | 19 | GAGACCCAGGGTAG | 20 | AAAAGTAATC |
| | | TGATTTGA | | GAAAGCCAAG |
| | | | | CAC |
| NEFL | 21 | ATGAGTTCCTTCAG | 22 | CTGGGCATCA |
| | | CTACGAGC | | ACGATCCAGA |
| CAMK2A | 23 | GCTCTTCGAGGAAT | 24 | CCTCTGAGAT |
| | | TGGGCAA | | GCTGTCATGT |
| | | | | AGT |
| ALB | 25 | CCTTTGGCACAATG | 26 | CAGCAGTCAG |
| | | AAGTGGGTAACC | | CCATTTCACC |
| | | | | ATAGG |
| CDX2 | 27 | CCTCTGAGATGCTG | 28 | GGTGATGTAG |
| | | TCATGTAGT | | CGACTGTAGT |
| | | | | GAA |
| AFP | 29 | TGTACTGCAGAGAT | 30 | CCTTGTAAGT |
| | | AAGTTTAGCTGAC | | GGCTTCTTGA |
| | | | | ACA |

KD of SIX2.

Gene KD was performed similar to as previously described (Velazco-Cruz et al., 2019). pLKO.1 TRC plasmids containing shRNA sequences targeting GFP (sh-ctrl) and human SIX2 (sh-SIX2-1 and sh-SIX2-2) were received from the RNAi Core at the Washington University. sh-ctrl, GCGCGATCACATGGTCCTGCT (SEQ ID NO: 35); sh-SIX2-1, CAACGAGAACTCCAATTCTAA (SEQ ID NO: 36); sh-SIX2-2, GAGCACCTTCACAAGAATGAA (SEQ ID NO: 37). Viral particles were generated using Lenti-X 293T cells (Takara; 632180) cultured in DMEM (MilliporeSigma; D6429) with 10% heat inactivated fetal bovine serum (MilliporeSigma; F4135). Confluent Lenti-X 293T cells were transfected with 6 pg of shRNA plasmid, 4.5 pg of psPAX2 (Addgene; 12260), and 1.5 μg pMD2.G (Addgene; 12259) packaging plasmids in 600 μL of Opti-MEM (Life Technologies; 31985-070) and 48 μL of Polyethylenimine ‘Max’ MW 40,000 Da (Polysciences; 24765-2). 16 hours post transfection media was switched. Viral containing supernatant was collected at 96 hours post transfection and concentrated using Lenti-X concentrator (Takara; 631232). Collected lentivirus was tittered using Lenti-X qRT-PCR Titration Kit (Takara; 631235). Lentiviral transduction occurred on the first day of Stage 6 by seeding 5 million dispersed single cells were into a well of a 6-well plate with lentivirus particles MOI of 5, media was switched 16 hours post transduction. psPAX2 and pMD2.G were a gift from Didier Trono.

Generation of SIX2 KO Cell Lines.

CRISPR/Cas9 genome engineering of the HUES8 cell line was performed by the Washington University Genome Engineering & iPSC Center. The high sequence similarity with other SIX genes limited the availability of high-quality gRNA sites, making conventional frameshift introduction infeasible. Instead, a deletion strategy was employed for almost all the SIX2 coding sequence. Unique genomic regions near the start and end of the SIX2 coding sequence were identified and guide RNAs were designed; 5′ gRNA, TCGGAGCTTCGTGGGACCCGCGG (SEQ ID NO: 38) and 3′gRNA, CCACGAGGTTGGCTGACATGGGG (SEQ ID NO: 39). Two homozygous SIX2 KO HUES8 cell lines were generated (KO-SIX2-1 and KO-SIX2-2). Validation of SIX2 KO was done by PCR using primers (GGGAGAACGAGTGAGAAGCG (SEQ ID NO: 31), TGCGGGTCTTTCAGTACCTG (SEQ ID NO: 32)) designed to amplify a 3368 bp sequence containing the coding region, deletion of which will produce a ˜300 bp amplicon (“Deletion primers”). Validation was also done using primers (CAGTTCTGGGAGAGAAGAGAC (SEQ ID NO: 33), GGGCTGGATTCTGTTCCCATA (SEQ ID NO: 34)) targeting within the SIX2 coding sequence designed to amplify 300 bp in wt and failing to amplify with successful deletion (“Inside primers”). Next generation sequencing was performed to further confirm KO.

Static Glucose-Stimulated Insulin Secretion.

Measurements were performed as previously described (Velazco-Cruz et al., 2019). The assay was performed in KRB buffer (128 mM NaCl, 5 mM KCl, 2.7 mM CaCl₂ 1.2 mM MgSO₄, 1 mM Na₂HPO₄, 1.2 mM KH₂PO₄, 5 mM NaHCO₃, 10 mM HEPES (GIBCO; 15630-080), and 0.1% BSA). Stage 6 clusters (˜20-30) were washed with KRB buffer and placed into transwells (Corning; 431752) with 2 mM glucose KRB. After 1 hr of equilibration, the solution was replaced with 2 mM glucose KRB for a 1 hr low glucose challenge, after which the solution was replace with 20 mM glucose alone or with 10 nM Extendin-4 (MilliporeSigma; E7144), 100 μM IBMX (MilliporeSigma; 15879), 300 μM Tolbutamide (MilliporeSigma; T0891), or 30 mM KCL (Thermo Fisher; BP366500) KRB for a 1 hr high glucose challenge. Incubations were performed in a humidified incubator at 37° C. 5% CO₂. Insulin was quantified with a human insulin ELISA (ALPCO; 80-INSHU-E10.1). Cell quantification was performed by dispersing with trypLE and counting with the Vi-Cell XR.

Dynamic Glucose-Stimulated Insulin Secretion.

Measurements were performed as previously described (Velazco-Cruz et al., 2019). Using an 8-channel peristaltic pump (ISMATEC; ISM931C) together with 0.015″ inlet/outlet two-stop tubing (ISMATEC; 070602-04i-ND) connected to 275-μl cell chamber (BioRep; Peri-Chamber) and dispensing nozzle (BioRep; PERI-NOZZLE) using 0.04″ connection tubbing (BioRep; Peri-TUB-040). Solutions, tubing, and cells were maintained at 37° C. using a water bath. Stage 6 clusters (˜20-30) were washed with KRB buffer and placed into perifusion cell chamber between two layers of hydrated Bio-Gel P-4 polyacrylamide beads (Bio-Rad; 150-4124). After 90 min of equilibration with 2 mM glucose KRB, cells were subjected to the following at 100 μL/min: 12 min of 2 mM glucose KRB, 24 min of 20 mM glucose KRB, and finally 12 min of 2 mM glucose KRB. Effluent was collected every 2 min. Insulin was quantified with a human insulin ELISA (ALPCO; 80-INSHU-E10.1). DNA quantification was performed by lysing the cells and measuring with the Quant-iT Picogreen dsDNA assay kit (Invitrogen; P7589). The lysis solution used consisted of 10 mM Tris (MilliporeSigma; T6066), 1 mM EDTA (Ambion; AM9261), and 0.2% Triton X-100 (Acros Organics; 327371000).

Immunostaining.

Measurements were performed as previously described (Velazco-Cruz et al., 2019). Stage 6 clusters were single-cell dispersed with trypLE, plated overnight, and fixed with 4% paraformaldehyde (Electron Microscopy Science; 15714) for 30 min at RT. Samples were treated for 30 min with blocking/permeabilizing/staining solution (5% donkey serum (Jackson Immunoresearch; 017-000-121) and 0.1% Triton X-100 (Acros Organics; 327371000) in PBS). Samples were then incubated overnight at 4° C. with primary antibody diluted in staining solution, incubated 2 hr at 4° C. with secondary antibodies diluted in staining solution, and stained with DAPI for 5 min. Nikon A1Rsi confocal microscope or Leica DMI4000 fluorescence microscope were used to take images. The antibodies used are listed in the methods section.

Flow Cytometry.

Measurements were performed as previously described (Velazco-Cruz et al., 2019). Clusters were single-cell dispersed with TrypLE, fixed with 4% paraformaldehyde for 30 min at 4° C., incubated 30 min at 4° C. in blocking/permeabilizing/staining solution, incubated with primary antibodies in staining buffer overnight at 4° C., incubated with secondary antibodies in staining buffer for 2 hr at 4° C., resuspended in staining buffer, and analyzed on an LSRII (BD Biosciences) or X-20 (BD Biosciences). Dot plots and percentages were generated using FlowJo. The antibodies used are listed in the methods section.

RNA Sequencing.

RNA from sh-ctrl and sh-SIX2-1 transduced cells (n=6 per condition) was extracted on Stage 6 Day 12 using RNeasy Mini Kit with DNase treatment. Washington University Genome Technology Access Center performed library preparation, sequencing, and determination of differential expression. Libraries were indexed, pooled, and single-end 50 base pair reads were sequenced on one lane of an Illumina HiSeq 3000 generating 25-30 million reads per sample. Reads were then aligned to the Ensembl release 76 top-level assembly with STAR. Gene counts were derived from the number of uniquely aligned unambiguous reads by Subread:featureCount. All gene counts were imported into EdgeR5 and TMM normalization size factors were calculated. Ribosomal genes and genes not expressed in the smallest group size minus one, samples greater than one count-per-million were excluded from further analysis. The TMM size factors and the matrix of counts were imported into Limma6. Weighted likelihoods based on the observed mean-variance relationship of every gene and sample were calculated for all samples with the voomWithQualityWeights7. Differential expression analysis was performed with a Benjamini-Hochberg false-discovery rate adjusted p values cut-off of less than or equal to 0.05. Hierarchical clustering and heatmaps were generated using Morpheus (https://software.broadinstitute.org/morpheus). To perform gene set enrichment analyses normalized all gene counts were imported into GSEA software and the Molecular Signature Database (MSigDB) Hallmark, KEGG, and Gene Ontology gene libraries were used to identify enriched gene sets (Subramanian et al., 2005). In Vitro Beta Cell Maturation gene set was generated by combining Veres et al. (Veres et al., 2019) Stage 6 enriched genes log base2 fold or greater (76 genes), and Nair et al. (Nair et al., 2019) β-clusters enriched genes (424 genes) with SIX2 removed.

Hormone Content Measurements.

Measurements were performed as previously described (Velazco-Cruz et al., 2019). Stage 6 cell clusters were collected, washed with PBS, placed in acid-ethanol solution (1.5% HCl and 70% ethanol), stored at −20° C. for 24 hours, vortexed, returned to −20° C. for 24 additional hours, vortexed, and centrifuged at 2100 G for 15 min. The supernatant was collected and neutralized with an equal volume of 1 M TRIS (pH 7.5). Human insulin and pro-insulin content were quantified using Human Insulin ELISA and Proinsulin ELISA (Mercodia; 10-1118-01) respectively. Samples were normalized to cell counts made using the Vi-Cell XR.

OCR and ECAR Measurements.

Measurements were performed similar to as previously reported (Millman et al., 2019). Stage 6 cell clusters were dispersed into a single-cell suspension and plated 200,000 per well. After overnight incubation in S6 media, the media was replaced with RPMI-1640 (Sigma; R6504) with 7.4 pH and 20 mM glucose. The Seahorse XFe24 flux analyzer (Agilent) was used to measure OCR and ECAR. After basal measurements, 3 μM oligomycin (Calbiochem; 1404-19-9), 0.25 μM carbonyl cyande-4-(trifluoromethoxy) phenylhydrazone (FCCP) (Sigma; 270-86-5), and 1 μM rotenone (Calbiochem; 83-79-4) and 2 μM antimycin A (Sigma; 1397-94-0) where injected sequentially and replicate measurements performed.

Cytoplasmic Calcium Measurements.

Calcium measurements were done similar as previously reported (Kenty and Melton, 2015, Pagliuca et al., 2014). Stage 6 day 12 clusters were single cell dispersed by incubation in TrypLE for 10 minutes and plated down onto a Matrigel coated #1.5 glass bottom 96 well plate (Cellvis; 963-1.5H-N) and allowed to attach overnight. Following overnight attachment clusters were washed twice with 2 mM glucose KRB and incubated in 2 mM glucose KRB with 20 μM Fluo-4 AM (Invitrogen; F14201) for 45 min at 37° C. Cells were washed twice with 2 mM glucose KRB, placed in 2 mM glucose KRB and incubated for 10 min at 37° C., then placed in a 37° C. 5% CO₂ humidified Tokai-hit stage-top on a Nikon Eclipse Ti inverted spinning disk confocal equipped with a Yokagawa CSU-X1 variable speed Nipkow spinning disk scan head equipped with a motorized xy stage including nano-positioning piezo z-insert. Images were acquired with the cells at 2 mM glucose KRB, 20 mM glucose KRB, and 20 mM glucose 30 mM KCl KRB. Analysis of calcium flux image stack was performed using ImageJ with the StackReg package for correction.

Quantification and Statistical Analysis.

GraphPad Prism was used to calculate statistical significance for all non-RNA sequencing data. One- or two-sided paired or unpaired t tests were used. Differential expression analysis of RNA sequencing data was performed with a Benjamini-Hochberg false-discovery rate adjusted p values cut-off of less than or equal to 0.05. Error bars represent s.e.m. unless otherwise noted. Sample size (n) is specified in each figure caption and indicates biological replicates unless otherwise noted. Statistical parameters are stated in figure captions.

### REFERENCES

Arda, H. E. E., Li, L., Tsai, J., Torre, E. A. A., Rosli, Y., Peiris, H., Spitale, R. C. C., Dai, C., Gu, X., Qu, K., et al. (2016). Age-Dependent Pancreatic Gene Regulation Reveals Mechanisms Governing Human b Cell Function. Cell Metab. 23, 909-920.
Balboa, D., Saarimäki-Vire, J., Borshagovski, D., Survila, M., Lindholm, P., Galli, E., Eurola, S., Ustinov, J., Grym, H., Huopio, H., et al. (2018). Insulin mutations impair beta-cell development in a patient-derived iPSC model of neonatal diabetes. eLife 7, e38519.
Bellin, M. D., Barton, F. B., Heitman, A., Harmon, J. V., Kandaswamy, R., Balamurugan, A. N., Sutherland, D. E. R., Alejandro, R., and Hering, B. J. (2012). Potent induction immunotherapy promotes long-term insulin independence after islet transplantation in type 1 diabetes. Am. J. Transplant. 12, 1576-1583.
Benner, C., van der Meulen, T., Caceres, E., Tigyi, K., Donaldson, C. J., and Huising, M. O. (2014). The transcriptional landscape of mouse beta cells compared to human beta cells reveals notable species differences in long non-coding RNA and protein-coding gene expression. BMC Genomics 15, 620.
Cardenas-Diaz, F. L., Osorio-Quintero, C., Diaz-Miranda, M. A., Kishore, S., Leavens, K., Jobaliya, C., Stanescu, D., Ortiz-Gonzalez, X., Yoon, C., Chen, C. S., et al. (2019). Modeling Monogenic Diabetes using Human ESCs Reveals Developmental and Metabolic Deficiencies Caused by Mutations in HNF1A. Cell Stem Cell 25, 273-289.e5.
D'Amour, K. A., Agulnick, A. D., Eliazer, S., Kelly, O. G., Kroon, E., and Baetge, E. E. (2005). Efficient differentiation of human embryonic stem cells to definitive endoderm. Nat. Biotechnol. 23, 1534-1541.
D'Amour, K. A., Bang, A. G., Eliazer, S., Kelly, O. G., Agulnick, A. D., Smart, N. G., Moorman, M. A., Kroon, E., Carpenter, M. K., and Baetge, E. E. (2006). Production of pancreatic hormone-expressing endocrine cells from human embryonic stem cells. Nat. Biotechnol. 24, 1392-1401.
Dwivedi, O. P., Lehtovirta, M., Hastoy, B., Chandra, V., Krentz, N. A. J., Kleiner, S., Jain, D., Richard, A.-M., Abaitua, F., Beer, N. L., et al. (2019). Loss of ZnT8 function protects against diabetes by enhanced insulin secretion. Nat. Genet. 51, 1596-1606.
Ghazizadeh, Z., Kao, D. I., Amin, S., Cook, B., Rao, S., Zhou, T., Zhang, T., Xiang, Z., Kenyon, R., Kaymakcalan, O., et al. (2017). ROCKII inhibition promotes the maturation of human pancreatic beta-like cells. Nat. Commun. 8, 298.
Gu, G., Dubauskaite, J., and Melton, D. A. (2002). Direct evidence for the pancreatic lineage: NGN3+ cells are islet progenitors and are distinct from duct progenitors. Development 129, 2447-2457.
Hachiya, T., Komaki, S., Hasegawa, Y., Ohmomo, H., Tanno, K., Hozawa, A., Tamiya, G., Yamamoto, M., Ogasawara, K., Nakamura, M., et al. (2017). Genome-wide meta-analysis in Japanese populations identifies novel variants at the TMC6-TMC8 and SIX3-SIX2 loci associated with HbA_{1c}. Sci. Rep. 7, 16147.
Hogrebe, N.J., Augsornworawat, P., Maxwell, K. G., Velazco-Cruz, L., and Millman, J. R. (2020). Targeting the cytoskeleton to direct pancreatic differentiation of human pluripotent stem cells. Nat. Biotechnol. 38, 460-470.
Hua, H., Shang, L., Martinez, H., Freeby, M., Gallagher, M. P., Ludwig, T., Deng, L., Greenberg, E., Leduc, C., Chung, W. K., et al. (2013). iPSC-derived b cells model diabetes due to glucokinase deficiency. J. Clin. Invest. 123, 3146-3153.
Kenty, J. H., and Melton, D. A. (2015). Testing pancreatic islet function at the single cell level by calcium influx with associated marker expression. PLoS One 10, e0122044.
Kim, Y. J., Go, M. J., Hu, C., Hong, C. B., Kim, Y. K., Lee, J. Y., Hwang, J.-Y., Oh, J. H., Kim, D.-J., Kim, N. H., et al.; MAGIC Consortium (2011). Large-scale genome-wide association studies in East Asians identify new genetic loci influencing metabolic traits. Nat. Genet. 43, 990-995.
Ma, S., Viola, R., Sui, L., Cherubini, V., Barbetti, F., and Egli, D. (2018). b Cell Replacement after Gene Editing of a Neonatal Diabetes-Causing Mutation at the Insulin Locus. Stem Cell Reports 11, 1407-1415.
Maxwell, K. G., Augsornworawat, P., Velazco-Cruz, L., Kim, M. H., Asada, R., Hogrebe, N.J., Morikawa, S., Urano, F., and Millman, J. R. (2020). Gene-edited human stem cell-derived b cells from a patient with monogenic diabetes reverse preexisting diabetes in mice. Sci. Transl. Med. 12, eaax9106.
McCall, M., and Shapiro, A. M. J. (2012). Update on islet transplantation. Cold Spring Harb. Perspect. Med. 2, a007823.
Millman, J. R., and Pagliuca, F. W. (2017). Autologous Pluripotent Stem Cell-Derived b-Like Cells for Diabetes Cellular Therapy. Diabetes 66, 1111-1120.
Millman, J. R., Xie, C., Van Dervort, A., Gurtler, M., Pagliuca, F. W., and Melton, D. A. (2016). Generation of stem cell-derived b-cells from patients with type 1 diabetes. Nat. Commun. 7, 11463.
Millman, J. R., Doggett, T., Thebeau, C., Zhang, S., Semenkovich, C. F., and Rajagopal, R. (2019). Measurement of Energy Metabolism in Explanted Retinal Tissue Using Extracellular Flux Analysis. J. Vis. Exp. (143) https://doi.org/10. 3791/58626.
Nair, G. G., Liu, J. S., Russ, H. A., Tran, S., Saxton, M. S., Chen, R., Juang, C., Li, M. L., Nguyen, V. Q., Giacometti, S., et al. (2019). Recapitulating endocrine cell clustering in culture promotes maturation of human stem-cell-derived b cells. Nat. Cell Biol. 21, 263-274.
Pagliuca, F. W., Millman, J. R., Gurtler, M., Segel, M., Van Dervort, A., Ryu, J. H.,
Peterson, Q. P., Greiner, D., and Melton, D. A. (2014). Generation of functional human pancreatic b cells in vitro. Cell 159, 428-439.
Rezania, A., Bruin, J. E., Arora, P., Rubin, A., Batushansky, I., Asadi, A., O'Dwyer, S., Quiskamp, N., Mojibian, M., Albrecht, T., et al. (2014). Reversal of diabetes with insulin-producing cells derived in vitro from human pluripotent stem cells. Nat. Biotechnol. 32, 1121-1133.
Romer, A. I., Singer, R. A., Sui, L., Egli, D., and Sussel, L. (2019). Murine Perinatal b-Cell Proliferation and the Differentiation of Human Stem Cell-Derived Insulin-Expressing Cells Require NEUROD1. Diabetes 68, 2259-2271.
Rosado-Olivieri, E. A., Anderson, K., Kenty, J. H., and Melton, D. A. (2019). YAP inhibition enhances the differentiation of functional stem cell-derived insulinproducing b cells. Nat. Commun. 10, 1464.
Rutter, G. A., and Hodson, D. J. (2013). Minireview: intraislet regulation of insulin secretion in humans. Mol. Endocrinol. 27, 1984-1995.
Segerstolpe, A., Palasantza, A., Eliasson, P., Andersson, E.-M., Andréasson, A.-C., Sun, X., Picelli, S., Sabirsh, A., Clausen, M., Bjursell, M. K., et al. (2016). Single-Cell Transcriptome Profiling of Human Pancreatic Islets in Health and Type 2 Diabetes. Cell Metab. 24, 593-607.
Shang, L., Hua, H., Foo, K., Martinez, H., Watanabe, K., Zimmer, M., Kahler, D. J., Freeby, M., Chung, W., LeDuc, C., et al. (2014). b-Cell dysfunction due to increased ER stress in a stem cell model of Wolfram syndrome. Diabetes 63, 923-933.
Spracklen, C. N., Shi, J., Vadlamudi, S., Wu, Y., Zou, M., Raulerson, C. K., Davis, J. P., Zeynalzadeh, M., Jackson, K., Yuan, W., et al. (2018). Identification and functional analysis of glycemic trait loci in the China Health and Nutrition Survey. PLoS Genet. 14, e1007275.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc. Natl. Acad. Sci. USA 102, 15545-15550.
Velazco-Cruz, L., Song, J., Maxwell, K. G., Goedegebuure, M. M., Augsornworawat, P., Hogrebe, N.J., and Millman, J. R. (2019). Acquisition of Dynamic Function in Human Stem Cell-Derived b Cells. Stem Cell Reports 12, 351-365.
Veres, A., Faust, A. L., Bushnell, H. L., Engquist, E. N., Kenty, J. H., Harb, G., Poh, Y. C., Sintov, E., G€urtler, M., Pagliuca, F. W., et al. (2019). Charting cellular identity during human in vitro b-cell differentiation. Nature 569, 368-373.
Xin, Y., Kim, J., Okamoto, H., Ni, M., Wei, Y., Adler, C., Murphy, A. J., Yancopoulos, G. D., Lin, C., and Gromada, J. (2016). RNA Sequencing of Single Human Islet Cells Reveals Type 2 Diabetes Genes. Cell Metab. 24, 608-615.

### Example 2: SIX2 Drives Stem-Cell Derived Insulin-Producing Beta Cell Functional Maturation

The following example describes how SIX2 drives stem-cell derived beta (SC-β) cell functional maturation and how increasing SIX2 activity or expression allows for glucose stimulated insulin secretion in SC-β cells.

Significance

Here it is shown that the acquisition of SIX2 gene and protein expression is necessary for SC-β cell function. The presence of SIX2 in SC-β cells improves several aspects of β cell function, such as glucose responsiveness, calcium coupling, mitochondrial respiration, insulin gene expression, insulin content, glucose-insulin coupling, and drives the maturation of the SC-β transcriptome.

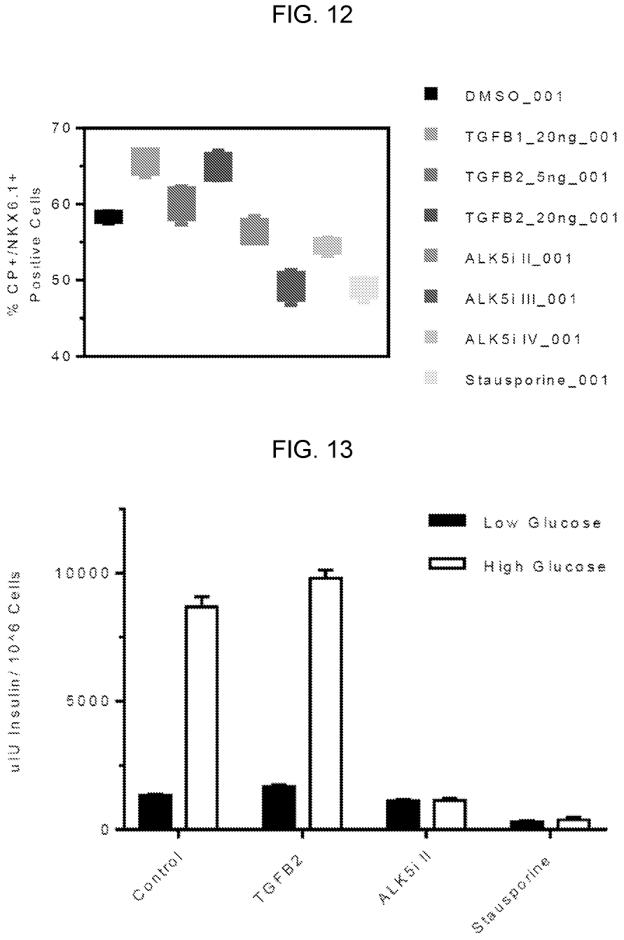

Only a fraction of C-peptide+ cells (SC-β cells) currently express SIX2 with current SC-β cell differentiation protocols (see e.g., FIG. 12). Increased co-expression results in differentiated populations with increased function and utility for cell therapy.

SIX2 expressing SC-β cells perform glucose stimulated insulin secretion when single cell dispersed and plated down in a similar fashion as SC-β clusters. This is significant in facilitating high throughput screens using SC-β cells.

### Background

The molecular mechanisms controlling human β cell functional maturation are not understood, preventing progressive improvements to SC-β cell technologies. Here it was shown that human specific gene SIX2 is necessary for acquisition of function in SC-β cells. SIX2 is the first gene shown to directly regulate SC-β cell functional maturation.

SC-β cell clusters are not amenable to high throughput screening. This hinders compound screens to increase functional maturation of SC-β cells. The inapplicability of SC-β cells to high throughput screening prevents their use for development of novel diabetes drug therapies.

As shown in Example 1, (1) SIX2 expression drives SC-β cell functional maturation (see e.g., FIG. 1A-FIG. 1C); (2) SIX2 is necessary for SC-β cell function, as determined by SIX2 knock out (KO) (see e.g., FIG. 1F-FIG. 1G); (3) SIX2 is necessary for SC-β cell function, as determined by SIX2 knock down (KD) (see e.g., FIG. 5); (4) SIX2 increases insulin gene expression and insulin content (see e.g., FIG. 4A, FIG. 4C); (5) SIX2 improves calcium signaling (see e.g., FIG. 4F); (6) SIX2 improves SC-β cell metabolic respiration (see e.g., FIG. 4D, FIG. 4E); (7) SIX2 improves insulin secretion in response to β cell secretagogues (see e.g., FIG. 4G); (8) SIX2 promotes maturation of SC-β cell transcriptome (see e.g., FIG. FIG. 3B, FIG. 3C, FIG. 3E); and (9) SIX2 regulates expression of beta cell functional maturation genes (see e.g., FIG. 7B).

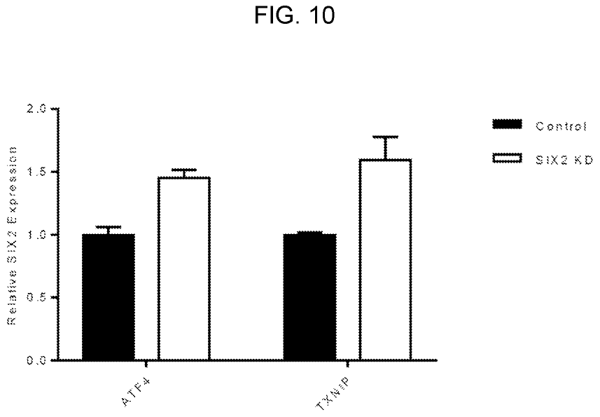

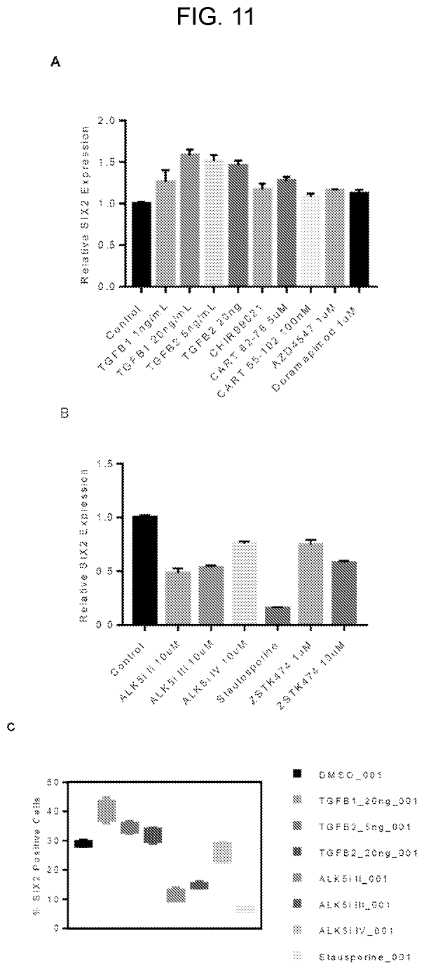

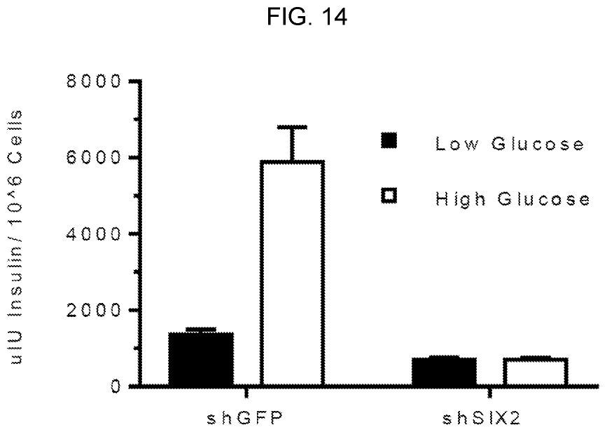

As shown in this example, SIX2 was shown to protect against cellular stress (see e.g., FIG. 10). Here, regulators of SIX2 gene expression were discovered (see e.g., FIG. 11). It was discovered that (1) regulators of SIX2 affect the % of SC-β cells generated (see e.g., FIG. 12); (2) SIX2 regulators influence SC-β cell function (see e.g., FIG. 13); (3) SIX2 is expressed by only a fraction of C-peptide positive cells (SC-β cells) (see e.g., FIG. 12A, FIG. 2B); and (4) SIX2+ cells secrete insulin in response to glucose when single cell dispersed and plated down (see e.g., FIG. 14).

### Example 3: SIX2 Regulates Beta Cell Health

Examples 1 and 2 described that SIX2, a transcription factor, was necessary for the functional maturation of insulin-secreting β cells. This example describes the additional discovery that SIX2 also affects β cell health. Without SIX2, beta cells are more susceptible to diabetes-associated stressors, such as inflammation, by having increased expression of stress response pathway genes and increased apoptosis.

Stem cell-derived beta cells (SC-β cells) are currently being commercialized for cell replacement therapy in diabetes. β cells become stressed upon transplantation. Targeting SIX2 could improve transplantation outcomes, such as by transient overexpression or chemically. β cell health and stress is central to the initiation and continuation of diabetes. Targeting SIX2 could provide for a novel anti-diabetic therapy.

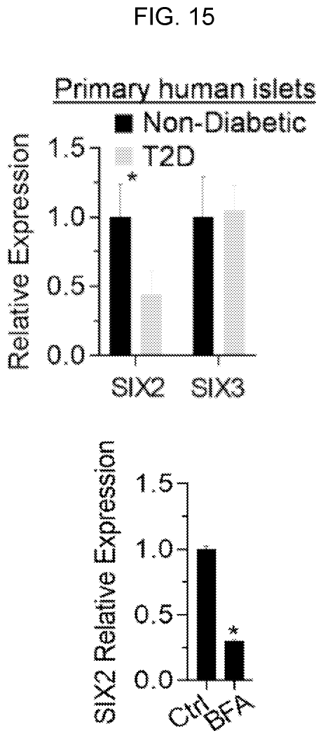

It has been shown here, that SIX2 expression is decreased in T2D islets and decreases in response to stress (see e.g., FIG. 15) as displayed in Type 2 diabetic human islets compared to nondiabetic human islets (see e.g., FIG. 15 (top)) and confirmed in SC-β cells with exogenous stress (see e.g., FIG. 15 (bottom)).

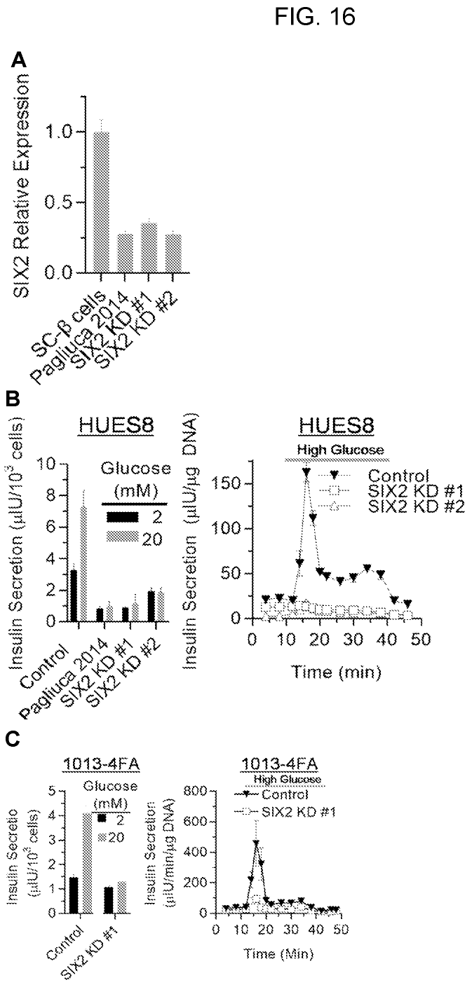

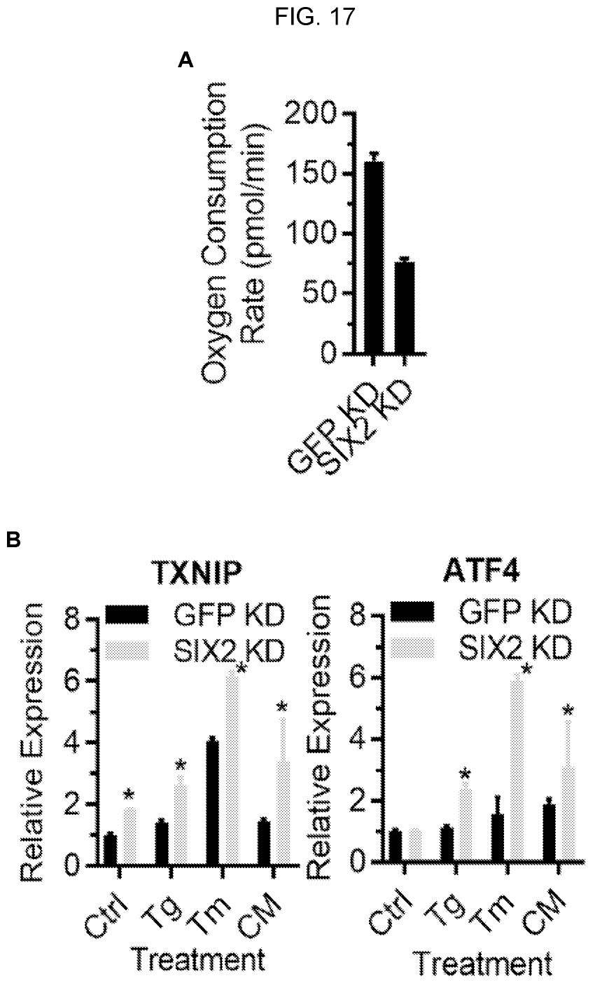

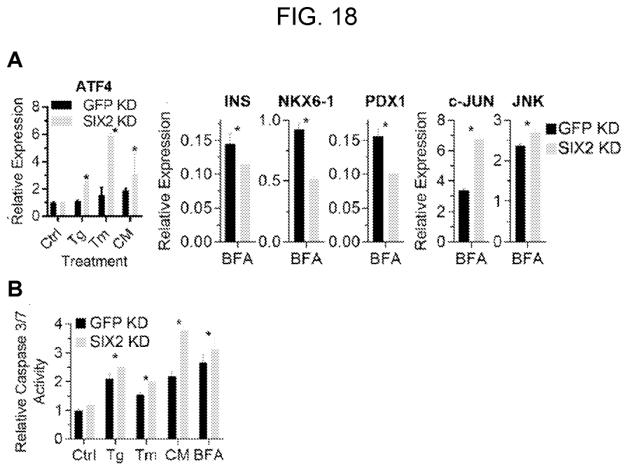

It was discovered here, that SIX2 is related to SC-β cell health. SIX2 knockdown reduces SC-β cell function (see e.g., FIG. 16A) and SIX2 knockdown reduces function in multiple other cell types (see e.g., FIG. 16B, FIG. 16C). Knockdown of SIX2 in SC-β cells reduces mitochondrial respiration (basal oxygen consumption rate levels, see e.g., FIG. 17A) and increases oxidative stress genes (TXNiP and ATF4) (see e.g., FIG. 17B). ER stress is increased with SIX2 KD in response to exogenous stress (see e.g., FIG. 18). SIX2 KD SC-β cells have increased ER stress with the addition of exogenous stress compared to GFP control, as shown by increased ER stress gene expression and reduced β cell gene expression (see e.g., FIG. 18). SIX2 KD SC-β are less resistant to ER-stress mediated cell death, as shown by increased Caspase 3/7 activation (see e.g., FIG. 18B). Caspase 3/7 is activated during apoptosis. HUES8 embryonic stem cell line was used to differentiate to stem cell-derived beta cells (SC-β cells). The lentiviruses to knockdown SIX2 gene expression were made in-house. SIX2 knockout cell lines from HUES8 were produced by the Genome Engineering and IPSC Center (GEiC).

Significance

The role of SIX2 in β cells is not well characterized, as it is not present in mouse β cells. SC-β cells allow for the study of SIX2 and its relation to function and stress. Data indicate reduction of SIX2 in diabetic human islets and SC-β cells with exogenous stress.

1. A method of generating SIX2-enhanced SC-β cells comprising:
providing a population of SC-β cells;
providing a SIX2 positive regulator; and
incubating the population of SC-β cells and the SIX2 positive regulator,
wherein the population of SC-β cells and the SIX2 positive regulator are incubated in an amount of the SIX2 positive regulator and for an amount of time sufficient to form an increased population of SIX2-enhanced SC-β cells or a population of SIX2-enhanced SC-β cells having increased SIX2 expression, function, or activity compared to the population of SC-β cells not in fluid contact with a SIX2 positive regulator.

2. The method of claim 1, wherein the population of SC-β cells are generated comprising the steps:
providing a stem cell;
providing serum-free media;
contacting the stem cell with a TGFβ/Activin agonist or a glycogen synthase kinase 3 (GSK) inhibitor or WNT agonist for an amount of time sufficient to form a definitive endoderm cell;
contacting the definitive endoderm cell with a FGFR2b agonist for an amount of time sufficient to form a primitive gut tube cell;
contacting the primitive gut tube cell with an RAR agonist for an amount of time sufficient to form an early pancreas progenitor cell;
incubating the early pancreas progenitor cell for at least about 3 days to form a pancreatic progenitor cell;
contacting the pancreatic progenitor cell with an Alk5 inhibitor, a gamma secretase inhibitor, SANT1, Erbb1 (EGFR) or Erbb4 agonist, or a RAR agonist for an amount of time sufficient to form an endocrine progenitor cell; and
allowing the endocrine progenitor (EP) cell to mature for an amount of time sufficient to form an SC-β cell.

3. The method of claim 1, wherein the effective amount of the SIX2 positive regulator results in
increased differentiation efficiency of the population of SC-β cells into mature SIX2-enhanced SC-β cells capable of biphasic insulin secretion in response to glucose;
SIX-2 enhanced SC-β cell exhibiting an increased fraction of C-peptide+ SC-β cells compared to the fraction of C-peptide+ SC-β cells not incubated with a SIX2 positive regulator;
the SIX2-enhanced SC-β cell exhibiting an increased fraction of C-peptide+/NKX6-1+ SC-β cells compared to the fraction of C-peptide+/NKX6-1+ SC-β cells not incubated with a SIX2 positive regulator;
improved glucose responsiveness compared to a SC-β cell not incubated with a SIX2 positive regulator;
improved calcium coupling compared to a SC-β cell not incubated with a SIX2 positive regulator;
improved mitochondrial respiration compared to a SC-β cell not incubated with a SIX2 positive regulator;
improved insulin gene expression compared to a SC-β cell not incubated with a SIX2 positive regulator;
improved insulin content compared to a SC-β cell not incubated with a SIX2 positive regulator;
improved glucose insulin coupling compared to a SC-β cell not incubated with a SIX2 positive regulator;
increased biphasic glucose stimulated insulin secretion compared to a SC-β cell not incubated with a SIX2 positive regulator;
increased glucose-stimulated insulin secretion compared to a SC-β ceil not incubated with a SIX2 positive regulator;
increased first and second phase insult secretion compared to a SC-β cell not incubated with a SIX2 positive regulator;
increased insulin gene expression and insulin content compared to a SC-6 cell not incubated with a SIX2 positive regulator;
decreased glucose stimulated calcium flux compared to a SC-β cell not incubated with a SIX2 positive regulator;
results in increased mitochondrial or metabolic respiration compared to a SC-β cell not incubated with a SIX2 positive regulator;
increased insulin secretion in response to β cell secretagogues compared to a SC-β cell not incubated with a SIX2 positive regulator;
improved β cell health compared to a SC-β cell not incubated with a SIX2 positive regulator;
decreased oxidative stress compared to a SC-β cell not incubated with a SIX2 positive regulator;
increased protection against cellular stress compared to a SC-β cell not incubated with a SIX2 positive regulator;
decreased endoplasmic reticulum (ER) stress compared to a SC-β cell not incubated with a SIX2 positive regulator; or
improved resistance to ER-mediated cell death compared to a SC-β cell not incubated with a SIX2 positive regulator.

4-23. (canceled)

24. The method of claim 1, wherein the SIX2 positive regulator promotes maturation of the SC-β cell transcriptome.

25. The method of claim 1, wherein the SIX2 positive regulator is selected from the group consisting of a TGFβ agonist, a glycogen synthase kinase 3 (GSK-3) inhibitor; a cocaine- and amphetamine-regulated transcript (CART) peptide fragment; an FGFR inhibitor; and a p38 MAPK inhibitor; and combinations thereof.

26. The method of claim 25, wherein
the TGFβ agonist is TGFβ1 or TGFβ2;
the GSK-3 inhibitor is CHIR99021;
the CART peptide fragment is CART 62-76 or CART 55-102;
the FGFR inhibitor is AZD4547; or
the p38 MAPK inhibitor is doramapimod.

27. The method of claim 2, wherein the stem cell is a HUES8 embryonic stem cell.

28. The method of claim 1, further comprising plating the SIX2-enhanced SC-β cells, wherein the SIX2-enhanced SC-β cells are passaged by single cell dispersion prior to plating.

29. The method of claim 28, wherein the plated single β cells are capable of glucose-stimulated insulin secretion.

30. The method of claim 1, further comprising transplanting the SIX2-enhanced SC-β cells into a subject in need thereof.

31. A method of treating a subject in need thereof comprising:
(i) administering or transplanting a therapeutically effective amount of SIX2-enhanced stem cell-derived beta cells (SC-β cells) to the subject;
(ii) transplanting SC-β cells to the subject, wherein SIX2 is activated after transplantation comprising administering a SIX2 positive regulator to the subject after the SC-β cells are transplanted into the subject; or
(iii) administering to a subject a therapeutically effective amount of a SIX2 positive regulator.

32. The method of claim 31, wherein the subject has diabetes.

33. The method of claim 31, wherein the subject has type 2 diabetes (T2D).

34-37. (canceled)

38. A method of screening comprising:
providing a SIX2-enhanced SC-β cell; and
introducing a compound or composition in fluid contact with the SIX2-enhanced SC-β cell, resulting in a treated SIX2-enhanced SC-β cell.

39. The method of claim 38, further comprising plating the SIX2-enhanced SC-β cells prior to introducing the compound or composition, wherein the SIX2-enhanced SC-β cells are passaged by single cell dispersion prior to plating.

40. (canceled)

41. A SIX2-enhanced SC-β cell having increased SIX2 expression, function, or activity compared to an SC-β cell not treated with a SIX2 positive regulator or not treated with a SIX2 positive regulator produced according to the method of claim 1.

42. The method of claim 2, further comprising contacting the primitive gut tube cell with a rho kinase inhibitor, a smoothened antagonist, a FGFR2b agonist, a protein kinase C activator, or a BMP type 1 receptor inhibitor for an amount of time sufficient to form an early pancreas progenitor cell.

43. The method of claim 2, further comprising contacting the early pancreas progenitor cell with a rho kinase inhibitor, a TGF-β/Activin agonist, a smoothened antagonist, an FGFR2b agonist, or a RAR agonist for an amount of time sufficient to form a pancreatic progenitor cell.

44. The method of claim 38, further comprising testing the SIX2-enhanced SC-β cell function or activity or measuring an amount of glucose stimulated insulin production.
